(19) United States
(12) Patent Application Publication
(10) Pub. No.: US 2025/0324955 A1
(43) Pub. Date: Oct. 23, 2025

(54) ZEBRAFISH MODEL OF HUMAN ACUTE MYELOID LEUKEMIA AND METHOD OF USE THEREOF

(71) Applicant: Versitech Limited, Hong Kong (CN)

(72) Inventors: Anskar Yu Hung LEUNG, Hong Kong (CN); Dandan WANG, Hong Kong (CN); Xuan SUN, Hong Kong (CN); Chun-hang Alvin MA, Hong Kong (CN); Yik Ling Bowie CHENG, Hong Kong (CN); Bailiang HE, Hong Kong (CN)

(21) Appl. No.: 18/873,308

(22) PCT Filed: Jun. 5, 2023
(86) PCT No.: PCT/CN2023/098302

§ 371 Date: Dec. 9, 2024

Related U.S. Application Data
(60) Provisional application No. 63/350,133, filed on Jun. 8, 2022.

(51) Int. Cl. A01K 67/0278

(52) U.S. Cl. CPC A01K 67/0278; A01K 2217/072; A01K 2227/40; A01K 2267/0331; A01K 2267/0393

(57) ABSTRACT

Genetically modified zebrafish, in which mutation combinations frequently identified in human AML are stably expressed in the stem cell population of the fish, are provided. The combination of mutations result in morphologic, cytochemical and molecular changes of its blood cells that are remarkably similar to those in human AML. The zebrafish model provides a foundation for the study of AML initiation and progression and a high throughput in vivo drug screening platform to identify personalized therapies for AML based on specific mutation combinations. The method of drug screening includes contacting embryos or adult fish containing mutations as disclosed herein, with a test agent, at test concentrations and test intervals to determine the therapeutic effect if any, of the test agent.

### FIELD OF THE INVENTION

The invention is generally directed to transgenic zebrafish, and more specifically related to transgenic zebrafish models that recapitulate pathologies associated with acute myeloid leukemia.

### BACKGROUND OF THE INVENTION

Cytogenetically normal acute myeloid leukemia (CN-AML) is a heterogeneous group of diseases sharing 20-30 recurrent mutations that are also seen in AML with abnormal cytogenetics, myelodysplastic syndrome (MDS) and myeloproliferative neoplasm (MPN). Each CN-AML carries on average 2-3 recurrent mutations, whose unique combinations give rise to the distinct clinicopathologic features and treatment outcome in individual patients.

Acute myeloid leukemia (AML) is one of the most lethal cancers worldwide. Recent advances in next-generation sequencing (NGS) underscore the genetic underpinning of this disease and reveal a diverse pattern of co-existing mutations that are associated with distinct molecular characteristics and clinical outcomes in different patients. Conventional chemotherapy and allogeneic hematopoietic stem cell transplantation (HSCT) are the mainstays of treatment but this “one-size fits all” approach has led to unsatisfactory patient outcome. Technologies allowing for the early detection of genetic alterations and understanding of these varied molecular pathologies have helped to advance our treatment regimens towards personalized targeted therapies. In spite of this, both AML and ALL continue to be a major cause of morbidity and mortality worldwide, in part because molecular therapies for the plethora of genetic abnormalities have not been developed. This underscores the current need for better model systems for therapy development.

There is an imperative need to develop disease models that address specific mutation combinations in AML for the study of their unique leukemic phenotypes and therapeutic response to treatment.

It is an object of the present invention to provide a zebrafish model for AML, with specific mutation combinations in human AML.

It is also an object of the present invention to provide method screening agents for their effect on AML.

### SUMMARY OF THE INVENTION

Provided herein are genetically modified zebrafish, in which mutation combinations frequently identified in human AML are stably expressed in the stem cell population of the fish, resulting in morphologic, cytochemical and molecular changes of its blood cells that are remarkably similar to those in human AML, herein after, zebrafish AML model. The zebrafish AML models have at least two mutations selected from the following as shown in Table 1.

TABLE 1
Gene mutations in zebrafish model of human AML

| Gene | Mutation |
|---|---|
| FLT3 | FLT3-ITD |
| NPM1 | NPMc+ |
| DNMT3A | DNMT3A-R882 |
| NRAS | NRAS-G12D |
| IDH2 | IDH2-R140Q, IDH2− R172K |
| TET2 | TET2−/− |
| RUNX1 | RUNX1−/− |
| IDH1 | IDH1-R132H |
| WT1 | WT1−/− |
| TP53 | TP53−/− |
| SRSF2 | SRSF2-P95H |
| ASXL1 | ASXL1−/− |
| STAG2 | STAG2−/− |
| PHF6 | PHF6−/− |

In a particularly preferred embodiment, the zebrafish AML model carries the specific mutation combinations FLT3^{ITD}+IDH2^{R140Q} or FLT3^{ITD}+IDH2^{R172K} that showed features of human AML carrying the same mutation combinations. In some forms the zebrafish AML model carries the specific mutation combinations SRSF2^{P95H} NRAS^{G12D}. In some forms the zebrafish AML model carries the specific mutation combinations asxl1^{+/-} IDH2^{R172K}. In some forms the zebrafish AML model carries the specific mutation combinations FLT3^{ITD}SRSF2^{P95H}. In some forms the zebrafish AML model carries the specific mutation combinations asxl1^{+/-} SRSF2^{P95H}.

Also provided are transgenic zebrafish lines expressing the specific mutation combinations FLT3^{ITD}IDH2^{R140Q} or FLT3^{ITD}IDH2^{R172K} in a cell- and tissue-specific manner.

In other specific embodiments Tg(mpo:EGFP, gata1:RFP, Runx1:FLT3^{ITD}IDH2^{R172K/R140Q}) and Tg(lyz:EGFP, corola:DsRED, Runx1:FLT3^{ITD}IDH2^{R172K/R140Q}) crossed zebrafish lines are provided. In general, the disclosed transgenic lines are crossed with lines expressing a tissue or cell type specific promoter operably linked to a reporter protein to obtain lines in which the disclosed combination of mutations are expressed in a cell or tissue specific manner dictated by the tissue or cell type specific promoter.

The zebrafish AML model provides a foundation for the study of AML initiation and progression and a high throughput in vivo drug screening platform to identify personalized therapies for AML based on specific mutation combinations. The method of drug screening includes contacting embryos or adult fish containing mutations as disclosed herein, with a test agent, at test concentrations and test intervals to determine the therapeutic effect if any, of the test agent.

### BRIEF DESCRIPTION OF THE DRAWINGS

FIGS. 1A-1E illustrate the generation and validation of the stable transgenic zebrafish lines with FLT3^{ITD} and IDH2 mutations. FIG. 1A is a schematic showing the syntenic neighboring genes (SNGs) of FLT3 and IDH2 in human and zebrafish. FIG. 1B is a graphical representation showing the protein sequence alignment of FLT3 between human (SEQ ID NO:7) and zebrafish (SEQ ID NO:8). FIG. 1C is schematic showing the protein sequence alignment of IDH2 between human (SEQ ID NO:9) and zebrafish (SEQ ID NO: 10). FIG. 1D are schematic diagrams of the Tol2 constructs of Runx1:FLT3^{ITD} and Runx1: IDH2^{140Q/172K}. FIG. 1E is a bar graph showing the expression of FLT3 and IDH2 mRNA in the KM of the stable transgenic zebrafish were confirmed via q-PCR.

FIG. 2A is a schematic illustration of the generation of transgenic embryos from stable transgenic adult zebrafish. FIG. 2B-2I are bar graphs showing the percentage of positively stained cmyb (FIG. 2B), runxl (FIG. 2C), mpo (FIG. 2D), l-plastin (FIG. 2E), SBB (FIG. 2F), ragl (FIG. 2g), gata1 (FIG. 2H), and pu.1 (FIG. 2I) in zebrafish embryos with FLT3^{ITD} and IDH2 mutations alone or in combination.

FIGS. 3A-3E are bar graphs demonstrating the development of AML-like disease in FLT3^{ITD} and IDH2 double mutant zebrafish. FIG. 3A shows the percentage of blast cells in the peripheral blood (PB) of transgenic zebrafish and WT siblings. FIG. 3B shows the percentage of blast cells in the kidney marrow (KM) of transgenic zebrafish and WT siblings. FIG. 3C shows the percentage of mpo+ cells in the KM in transgenic zebrafish compared with WT siblings. FIG. 3D shows the relative size of KM in transgenic zebrafish compared with wildtype siblings. FIG. 3E shows the percent of erythroid (R1), myeloid (R2), lymphoid/HSPCs (R3) and precursors (R4) in the KM of transgenic zebrafish compared to the parental lines.

FIGS. 4A-4E are graphs demonstrating the effect of FLT3^{ITD} and IDH2 mutations on the induction of AML-like phenotypes in zebrafish. FIG. 4A is a bar graph showing the total cell number in spleens of FLT3^{ITD} and IDH2 double mutant and wildtype siblings. FIG. 4B is a bar graph showing the percentage of myelomonocytes in spleens of FLT3^{ITD} and IDH2 double mutant and wildtype siblings. FIG. 4C is a survival curve illustrating the overall survival of wildtype, single mutant, and double mutant zebrafish. FIG. 4D is a bar graph showing the body weight of mutant zebrafish and wildtype siblings. FIG. 4E is a bar graph showing the total kidney marrow cellularity of mutant zebrafish and wildtype siblings.

FIG. 5A is a schematic illustration for the generation of mutant zebrafish in Tg(rag2:EGFP) background. FIG. 5B is a bar graph showing the relative expression of B cell, T cell, HSPC markers and IDH2 in the thymus of IDH2^{R172K} and wildtype zebrafish. FIG. 5C is a sequence chromatograph illustrating confirmation of the presence of IDH2 hotspot mutations in the KM and thymus of the mutant fish via sanger sequencing.

FIG. 6A is a schematic representation of the transplantation procedure. FIG. 6B is a survival plot of the irradiated recipients 40 days post primary transplantation. FIG. 6C-6E are representative flow cytometric dot plots for erythroid (R1), myeloid (R2), lymphoid/HSPCs (R3) and precursors (R4) analysis of KM from recipients transplanted with wildtype or mutant marrow. FIG. 6F is a bar graph showing the percentage of R3 and R4 in the KM of the primary recipients. FIG. 6G is a bar graph showing the relative mRNA expression of FLT3 and IDH2 in kidney marrow of the recipients 30 days post-transplantation. FIG. 6H is a bar graph showing the total cell number of kidney marrow cells in the recipients 30 days post-transplantation. FIG. 6I is a bar graph showing the percentage of blast cells in whole kidney marrow of the recipients 30 days post-transplantation. FIG. 6J is a bar graph showing the percentage of blast cells in the peripheral blood (PB) of the recipients 30 days post-transplantation. FIG. 6K is a bar graph showing the spleen sizes of recipients 30 days post-transplantation. FIG. 6L is a bar graph showing the percentage of blast cells and macrophages in the spleens of recipients 30 days post-transplantation. FIG. 6M is a survival plot of irradiated secondary recipients 40 days post-transplantation.

FIG. 7A is a uniform manifold approximation projection (UMAP) plot illustrating the 16 cell types identified based on key hematopoietic marker genes in zebrafish, murine, and human. FIG. 7B is a dot plot showing the proportions of distribution between double mutant and wildtype whole kidney marrow cells in different clusters. FIG. 7C-7F are volcano plots showing the differentially expressed genes in HSC (FIG. 7C), myeloid progenitor (FIG. 7D), erythroid progenitor (FIG. 7E) and HSPC-MPP (FIG. 7F) from Tg(Runx1:FLT3^{ITD}IDH2^{R140Q}) and wildtype whole kidney marrow. FIG. 7G is a volcano plot showing the differentially expressed genes in HSPC-MPP from Tg(Runx1:FLT3^{ITD}IDH2^{R172K}) and WT WKM. Genes with significant enrichment are shown as red or blue (log 2 fold change >0.25 or <−0.25 respectively).

FIGS. 8A and 8B are dot plots showing results from pathway analyses of upregulated pathways in FLT3^{ITD} and IDH2 double mutant zebrafish. FIG. 8A is a dot plot showing the differentially enriched hallmark terms within the 16 cell clusters between Tg(Runx1:FLT3^{ITD}IDH2^{R140Q}) and wildtype. FIG. 8B is a dot plot showing the differentially enriched hallmark terms within the 16 cell clusters between Tg(Runx1:FLT3^{ITD}IDH2^{R172K}) and wildtype. FIGS. 8C-8G are violin plots showing expression of pcna (FIG. 8C), chac1 (FIG. 8D), cox7c (FIG. 8E), rps7 (FIG. 8F), mhc1uba (FIG. 8G) and rps8a (FIG. 8H) across different cell clusters between double mutant and wildtype zebrafish.

FIGS. 9A-9C are bar graphs showing the response of the FLT3^{ITD} and IDH2 double mutant zebrafish to therapeutic treatments. FIGS. 9A-C show the results from quantitative analyses of the SSB+ neutrophils in FLT3^{ITD} and IDH2 double mutant presented as a percentage relative to untreated control embryos following in vivo treatment with gilteritinib (FIG. 9A), quizartinib (FIG. 9B), and enasidenib (FIG. 9C) at various concentrations (0.01 μM, 0.1 μM and 1 μM). FIGS. 9D and 9E are survival plots showing the sensitivity of wildtype zebrafish to quizartinib (FIG. 9D) or enasidenib (FIG. 9E) 14 days post treatment. FIG. 9F is a schematic diagram of drug administration in adult zebrafish via oral gavage. FIG. 9G is a bar graph showing changes in total kidney marrow cellularity in adult FLT3^{ITD} and IDH2 double mutant and wildtype zebrafish post-treatment. FIGS. 9H-9K are bar graphs showing the percentage of blast cells (FIG. 9H), neutrophils (FIG. 9I), erythroid cells (FIG. 9J) and total number of kidney marrow cells (FIG. 9K) in whole kidney marrow post-treatment. FIG. 9L is a bar graph showing quantification of the size of the spleen in FLT3^{ITD} and IDH2 double mutant relative to wildtype zebrafish post-treatment with quizartinib and/or enasidenib.

FIG. 10A-10H show the development of CMML or AML-like disease in SRSF2 and NRAS mutant zebrafish. FIG. 10A. Representative Wright and Giemsa staining of the peripheral blood (PB), Kidney marrow (KM) cells and spleen cells, and Nonspecific esterase (NSE) staining of the KM cells FIG. 10B) from the mutant zebrafish and WT siblings. FIG. 10C. The percentage of the blast cell in the PB of the transgenic mutant zebrafish and WT siblings. FIG. 10D. The total cell number, the percentage of blast cell (FIG. 10E), the percentage of NSE+ cells (FIG. 10F) in the KM of the transgenic mutant zebrafish compared with WT siblings. FIG. 10G. Relative mRNA expression of SRSF2 and NRAS in the KM of the transgenic mutant zebrafish and wildtype zebrafish. FIG. 10H. Overall survival of the WT, single mutant, and double mutant. Data are mean±s.e.m. One-way Anova was performed for FIGS. 10C, 10D, 10E, 10F and 10G, *P<0.05, **P<0.01, ****P<0.0001. Log-Rank test was performed for FIG. 10H, *P<0.05.

FIG. 11A-11B: Cytospin analysis of 9-month-old transgenic line carrying asxl1 and IDH2^{R172K} mutations. (FIG. 11A) Representative Wright-Giemsa staining of cytospin of kidney marrow (KM) of the control, single and double mutant transgenic line. (FIG. 11B) The percentage of the erythroid, neutrophils, lymphocytes monocytes/macrophages, eosinophil, myeloid precursors, immature monocytes and blast-like cells from the KM was quantified. Statistical analysis was performed using the Student's t test, p<0.05 was considered to be statistically significant.

FIG. 12A-12B. Cytospin analysis of 6-month-old transgenic line carrying asxl1+/− and SRSF2P95H mutations. (FIG. 12A) Representative Wright-Giemsa staining of peripheral blood smear, cytospin of kidney marrow, and nonspecific esterase staining (NSE) of kidney marrow of the control, single and double mutant transgenic line. (FIG. 12B) The percentage of the erythroid, neutrophils, lymphocytes, blast-like cells, myeloid precursors, eosinophils, mature monocytes, immature monocytes, and NSE+ cells from the KM was quantified. Statistical analysis was performed using the Student's t test, p<0.05 was considered to be statistically significant.

FIG. 13A-13B. Cytospin analysis of 1-year-old transgenic line carrying FLT3ITD and SRSF2P95H mutations. (FIG. 13A) Representative Wright-Giemsa staining of peripheral blood smear, cytospin of kidney marrow, and nonspecific esterase staining (NSE) of kidney marrow of the control, single and double mutant transgenic line. (FIG. 13B) The percentage of the erythroid, neutrophils, lymphocytes, blast-like cells, myeloid precursors, eosinophils, mature monocytes, immature monocytes, and NSE+ cells from the KM was quantified. Statistical analysis was performed using the Student's t test, p<0.05 was considered to be statistically significant.

### DETAILED DESCRIPTION OF THE INVENTION

The disclosed zebrafish AML model is specifically designed to address the issues pertaining to the diversity of mutation profiles in AML that has hampered the development of personalized treatment of these patients. Specifically, the burgeoning information about the genetic landscape of AML underscores the need for robust and innovative models that can delineate the roles of recurring mutations and their combinations in leukemogenesis and inform treatment for individual patients at high throughput. Zebrafish models carrying specific mutation combinations for example FLT3^{ITD} IDH2^{R140Q} and FLT3^{ITD}IDH2^{R172K} showed features of human AML carrying the same mutation combinations.

The disclosed zebrafish AML model is distinct from prior attempts at zebrafish models. For example, CN 103977424 discloses a stable transgenic zebrafish in which a point mutation was generated within the pu.1 gene (pu.1G242D), and this mutation has led to a reduction of Pu.1 activity. The reduced Pu.1 activity resulted in an increased abundance of immature myeloid cells in the transgenic zebrafish embryos and the expansion of myeloid blasts in adult zebrafish kidney marrow. US 2009/0055940 discloses provided a stable transgenic zebrafish line that expressed the human AMLI-ETO fusion gene from an inducible promoter (Hsp70). Induction of AMLI-ETO expression caused a block in hematopoietic maturation and accumulation of immature hematopoietic progenitors in the intermediate cell mass (ICM) and a concomitant loss of circulating cells. These phenotypes were readily detected in the intact zebrafish embryo within two days of fertilization. US 2004/0117867 discloses transgenic fish whose genome has stably-integrated a mouse oncogene (C-myc) that was linked to a zebrafish lymphoid-specific promoter (rag2) and the oncogene was shown to induce T-cell lymphoma or a T-cell acute lymphoblastic leukaemia. Xu, et al., vol. 105 No. 3 (2020): March 2020 https://doi.org/10.3324/haematol.2019.215939, discloses a genetically modified zebrafish model with stable expression of human BCR/ABL1 oncoprotein to elucidate the mechanisms of CML disease progression; however, the mutations involved, and disease of interest were completely different than disclosed herein. Gjini, et al., Dis Model Mech. 2019 May 7; 12 (5): dmm035790. doi: 10.1242/dmm.035790, discloses a genetically modified zebrafish model with loss-of-function mutation of asxl1gene. The combined loss of asxl1 and tet2 resulted in the development of a more penetrant MPN phenotype and AML in adult transgenic zebrafish. The disclosed zebrafish resulted from a different genome editing strategy and focused on the stable lineage-specific expression of combinations of gain of function AML mutations, of combinations of genes as shown in Table and showed features of human AML at embryonic and adult stages. Forrester, et al., British Journal of Haematology, 155,167-181 (2011) disclose an inducible transgenic zebrafish harbouring human NUP98-HOXA9 fusion gene under the control of a lineage-specific promoter (pu.1) and induced MPN-like phenotypes in both embryonic and adult stages of the transgenic zebrafish. Bolli, et al.; 115 (16): 3329-40 discloses a zebrafish model in which human NPM mutant (NPMc+) was transiently overexpressed in the zebrafish embryos and caused an increase in the number of myeloid progenitors. Zhuracleva, et al., British Journal of Haematology, 2008, 143 (3): 378-382. Disclose a transgenic zebrafish in which the human MYST3/NCOA2 fusion gene was expressed under the control of a lineage specific promoter (pu.1) and demonstrated the oncogenic potential of the MYST3/NCOA2 fusion protein in vivo.

By contrast, the disclosed zebrafish AML model is on a different and more efficient genome editing strategy and focused on modelling combinations of AML-associated mutations (at least two genes from Table 1) and preferably do not incorporate the specific mutations discussed above for example, no transient expression of NPMc+, no combined loss of asxl1 and tet2, no point mutation within the pu.1 gene, no expression the discussed fusion genes-MYST3/NCOA2 fusion gene or AMLI-ETO fusion gene, no stable expression of human BCR/ABL1, etc.

### I. Definitions

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The terms “dpf” and “hpf” refer to the natural law of “after fertilization”. For example, “3 dpf” refers to after fertilization three days, and “8 hpf” refers to after fertilization 8 hours.

The term “transgenic” refers to an organism and the progeny of such an organism that contains a nucleic acid molecule that has been artificially introduced into the organism. As used herein, “transgenic zebrafish” refers to zebrafish, or progeny of zebrafish into which an exogenous construct has been introduced.

The term “variant” or “mutant,” as used herein refer to an artificial outcome that has a pattern that deviates from what occurs in nature.

The terms “vector” or “expression vector” refer to a system suitable for delivering and expressing a desired nucleotide or protein sequence. Some vectors may be expression vectors, cloning vectors, transfer vectors etc.

The term “reporter protein” refers to any protein that can be specifically detected when expressed.

As used herein, the term “expressing” in relation to a gene or protein refers to making an mRNA or protein which can be observed using assays such as microarray assays, antibody staining assays, and the like.

As used herein, the term “differentially expressed” as applied to a gene, refers to the production of the mRNA transcribed from the gene, or the protein product encoded by the gene that is different compared to normal or control. A differentially expressed gene may be overexpressed or underexpressed as compared to the expression level of a normal or control cell. In one aspect, it refers to a differential that is at least 1.5 times, or at least 2.5 times, or alternatively at least 5 times, or alternatively at least 10 times higher or lower than the expression level detected in a control sample. The term “differentially expressed” also refers to nucleotide sequences in a cell or tissue which are expressed where silent in a control cell or not expressed where expressed in a control cell.

The term “gene cloning” or “DNA cloning” refers to assembling recombinant DNA molecules and directing their replication in a host organism.

As used herein, the term “marker” or “cell marker” refers to gene or protein that identifies a particular cell or cell type. A marker for a cell may not be limited to one marker, markers may refer to a “pattern” of markers such that a designated group of markers may identity a cell or cell type from another cell or cell type.

The term “founder fish” refers to the transformed zebrafish containing the germ cells, in which the transgenic construct was integrated into the genome. The progeny derived from these transgenic germ cells in the F1 generation will have the tissue specific expression of a reporter protein, for example, EGFP expressed in the eye.

The term “proliferation” refers to an increase in cell number.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

### II. Zebrafish Model of Human AML

Zebrafish has emerged as a model organism for the study of human diseases including leukemia. The optical transparency and high fecundity are distinct advantages, and the zebrafish genome and hematopoietic system are remarkably similar to those in mice and human. Moreover, recent advances in genome editing and transgenesis and the rapid embryonic development have made zebrafish a unique model for the study of mutation combinations at high throughput. Due to the relatively small size and external development of zebrafish embryos and larvae, growth is possible in small dishes, enabling multiple growth conditions amenable to drug screens. Furthermore, during early stages of their development, zebrafish are translucent, allowing for direct observation of distinct cells and tissues in real-time (such as with fluorescent transgenes).

The disclosed zebrafish model harnesses the cooperativity of mutation combinations disclosed in Table 1, which is different from various zebrafish models previously disclosed, and which result in zebrafish showing features of human AML at embryonic and adult stages. For example, US 2009/0055940 discloses a zebrafish model of MLL (Mixed Lineage Leukemia; Myeloid Lymphoid Leukemia) in which endogenous zebrafish mll gene (leukemia related gene) was transiently knocked down. WO 2007/014318 discloses a transgenic zebrafish line that expressed the human AMLI-ETO fusion gene from an inducible promoter (Hsp70). US 2004/0117867 discloses transgenic fish whose genome integrates a mouse oncogene (C-myc) that was linked to a zebrafish lymphoid-specific promoter (rag2). CN 103977424 discloses transgenic zebrafish in which a point mutation was generated within the pu.1 gene (pu.1G242D). Others disclosures similarly target different genes, for example, human BCR/ABL1 (X U, et al., Hematologica, 105 (3): 674-686 2020), loss-of-function mutation of asxl1gene (Gjini, et al., Disease Models & Mechanisms, 12 (5): dmm035790 2019); transgenic zebrafish harboring human NUP98-HOXA9 fusion gene under the control of a lineage-specific promoter (pu.1) (Forrester, et al., British Journal of Hematology, 155:167-181 (2011)); a zebrafish model in which human NPM mutant (NPMc+) was transiently overexpressed in the zebrafish embryos (Bolli, et al., Blood, 115 (16): 3329-40 (2010)); a transgenic zebrafish in which the human MYST3/NCOA2 fusion gene was expressed under the control of a lineage specific promoter (pu.1) (Zhuravleva, et al., British Journal of Hematology, 143 (3): 378-82 (2008)).

By contrast, the zebrafish model provided herein is based on a different and more efficient genome editing strategy selecting combinations of AML-associated mutations, thus providing a zebrafish model showing features of human AML at embryonic and adult stages.

### A. Combinations of Gene Mutations

Provided are transgenic zebrafish lines that express two or more human gene mutations associated with Acute Myeloid Leukemia (AML). The disclosed transgenic zebrafish lines contain a combination of two or more AML-associated mutations in the genes, fms related tyrosine kinase 3 (FLT3), nucleophosmin 1 (NPM1), dna methyltransferase 3 alpha (DNMT3a), nras proto-oncogene, gtpase (NRAS), isocitrate dehydrogenase 2 (IDH2), tet methylcytosine dioxygenase 2 (TET2), runx family transcription factor 1 (RUNX1), isocitrate dehydrogenase 1 (IDH1), wilm's tumor transcription factor (WT1), tumor protein p53 (TP53), serine and arginine rich splicing factor 2 (SRSF2), asxl transcriptional regulator 1 (ASXL1), stromal antigen 2 (STAG2), and PHD finger protein 6 (PHF6). Specifically, the disclosed zebrafish model may contain two or more of the following AML-associated gene mutations: FLT3^{ITD}, NPMc+, DNMT3A^{R882}, NRAS^{G12D}, IDH2^{R140Q}, IDH2^{R172K}, TET2^{-/-}, RUNX1^{-/-}, IDH1^{R132H}, WT1^{-/-}, TP53^{-/-}, SRSF2^{P95H}, ASXL^{-/-}, STAG2^{-/-}, and PHF6^{-/-} (−/− is used in reference to a gene to refer to homozygous negative for the gene).

Acute myeloid leukemia with a FLT3 internal tandem duplication (FLT3/ITD) mutation is an aggressive hematologic malignancy with a generally poor prognosis (reviewed in Levis, Blood, 117 (26): 6987-6990 (2011). The nucleophosmin (NPM1) gene encodes for a multifunctional nucleocytoplasmic shuttling protein that is localized mainly in the nucleolus. NPM1 mutations occur in 50% to 60% of adult acute myeloid leukemia with normal karyotype (AML-NK) and generate NPM mutants that localize aberrantly in the leukemic-cell cytoplasm, hence the term NPM-cytoplasmic positive (NPMc+AML (Falini, et al., Blood, 109 (3): 874-85 (2007). The DNMT3A mutation is a common genetic aberration in AML patients. The most common mutation is located in codon R882 (DNMT3AR882mut) (Blau, et al., Blood, 132 (Suppl1); 5263 (2018).

In some embodiments, the zebrafish model contains 2, 3, 4, 5, 6, 7, 8, 9, 10, 11, 12, or 13 of the following AML-associated gene mutations: FLT3^{ITD} (FLT3 internal tandem duplication), NPMc+, DNMT3α^{R882}, NRAS^{G12D}, IDH2^{R140Q}, IDH2^{R172K}, TET2^{-/-}, RUNX1^{-/-}, IDH1^{R132H}, WT1^{-/-}, TP53^{-/-}, SRSF2^{P95H}, ASXL^{-/-}, STAG2^{-/-}, and PHF6^{-/-}. Preferably, the zebrafish model contains 2-5 of the following AML-associated gene mutations: FLT3^{ITD}. NPMc+, DNMT3A^{R882}, NRAS^{G12D}, IDH2^{R140Q}, IDH2^{R172K}, TET2^{-/-}, RUNX1^{-/-}, IDH1^{R132H}, WT1^{-/-}, TP53^{-/-}, SRSF2^{P95H}, ASXL^{-/-}, STAG2^{-/-} and PHF6^{-/-}. In a preferred embodiment, the transgenic zebrafish model contains the AML-associated mutations FLT3^{ITD} and IDH2^{R172K}. In another preferred embodiment, the transgenic zebrafish model contains the AML-associated mutations FLT3^{ITD} and IDH2^{R140Q}. In another preferred embodiment, the transgenic zebrafish model contains the AML-associated mutations FLT3^{ITD} and IDH2^{R140Q/172K}.

Also disclosed are transgenic zebrafish models containing tissue and/or cell type specific AML-associated mutations. The tissue and/or cell type specific mutations may be in two or more of the following genes: fms related tyrosine kinase 3 (FLT3), nucleophosmin 1 (NPM1), DNA methyltransferase 3 alpha (DNMT3a), nras proto-oncogene, gtpase (NRAS), isocitrate dehydrogenase (nadp⁺) 2 (IDH2), tet methylcytosine dioxygenase 2 (TET2), runx family transcription factor 1 (RUNX1), isocitrate dehydrogenase (NADP⁺)1 (IDH1), wilm's tumor 1 (WT1) transcription factor (WT1), tumor protein p53 (TP53), serine and arginine rich splicing factor 2 (SRSF2), asxl transcriptional regulator 1 (ASXL1), stromal antigen 2 (STAG2), and PHD finger protein 6 (PHF6). Specifically, the tissue and/or cell type specific AML-associated gene mutations may be one or more of the following mutations: FLT3^{ITD}, NPMc+, DNMT3A^{R882}, NRAS^{G12D}, IDH2^{R140Q}, IDH2^{R172H}, TET2^{-/-}, RUNX1^{-/-}, IDH1^{R132H}, WT1^{-/-}, TP53^{-/-}, SRSF2^{P95H}, ASXL^{-/-}, STAG2^{-/-}, and PHF6^{-/-}. Preferably, the transgenic zebra fish models containing the tissue and/or cell type specific AML-associated gene mutations are the zebrafish lines, Tg(mpo:EGFP, gata1:RFP, Runx1: FLT3^{ITD}IDH2^{R172K/R140Q}) and Tg(lyz:EGFP, corola:DsRED, Runx1: FLT3^{ITD}IDH2^{R172K/R140Q}).

FLT3 is a transmembrane ligand-activated receptor tyrosine kinase that is normally expressed by hematopoietic stem or progenitor cells and plays an important role in the early stages of both myeloid and lymphoid lineage development. An extracellular ligand (FLT3 ligand) binds and activates FLT3, promoting cell survival, proliferation, and differentiation through various signaling pathways, including PI3K, RAS, and STAT5. Mutations of FLT3 are found in approximately 30% of newly diagnosed AML cases and occur as either ITDs (≈25%) or point mutations in the TKD (7-10%). FLT3-ITD occurs in the form of a replicated sequence in the juxtamembrane domain and/or TKD1 of the FLT3 receptor and varies in location and length within these domains. Both FLT3-ITD and FLT3-TKD mutations constitutively activate FLT3 kinase activity, resulting in proliferation and survival of AML (Reviewed in Daver, et al., Leukemia, 33:299-312 (2019).

Approximately one third of acute myeloid leukemias (AMLs) are characterized by aberrant cytoplasmic localization of nucleophosmin (NPMc+AML), consequent to mutations in the NPM putative nucleolar localization signal. The mutations, collectively termed NPMc+, cluster at the 3′ end of the NPM1 open reading frame and introduce a nuclear export signal that causes relocalization of nucleophosmin from the nucleolus to the cytoplasm. Patel et al., Curr Hematol Malig Rep. 2020; 15 (4): 350-359.

DNMT3A is a kind of methyltransferase that is responsible for the de novo methylation of CpG dinucleotides. DNMT3A is crucial for the establishment and maintenance of cellular methylation patterns. DNMT3A mutation is a common genetic aberration in AML patients. The most common mutation is located at codon R882 (DNMT3AR882).

### B. Characteristics of Transgenic Zebrafish

Provided are transgenic zebrafish lines that exhibit pathological features associated with Human Acute Myeloid Leukemia (AML). The AML-related pathological features may be cellular, cytochemical and/or molecular.

### 1. Morphological Characteristics

The disclosed transgenic zebrafish can exhibit one or more morphological features consistent with human AML. The transgenic zebrafish may exhibit lower weight and decreased survival. The transgenic zebrafish may demonstrate changes in splenic and kidney marrow structure, including increases in size of the spleen and kidney marrow. The transgenic zebrafish may or may not have an enlarged thymus.

### 2. Cellular Characteristics

The disclosed transgenic zebrafish demonstrate one of more cellular features consistent with human AML. The transgenic zebrafish may have increased circulating blasts and MPO+ myeloblasts; increased myeloid precursors, erythroid precursors, and lymphocytes; increased number of macrophages and myeloid cells and binucleated erythroid cells as well as dysplastic neutrophils may be observed. Blasts are precursors to the mature, circulating blood cells such as neutrophils, monocytes, lymphocytes and erythrocytes. Blasts are usually found in low numbers in the bone marrow. They are not usually found in significant numbers in the blood. Blasts tend to be medium to large cells with a large nucleus that takes up most of the cell (high nuclear:cytoplasmic ratio), fine nuclear chromatin pattern, nucleoli and a grey to blue cytoplasm. No single characteristic identifies a blast. In general, blasts are cells that have a large nucleus, immature chromatin, a prominent nucleolus, scant cytoplasm and few or no cytoplasmic granules. Blasts may not have all of these features. Cell size-blasts are often medium to large cells. They are usually larger than a lymphocyte and at least the size of a monocyte.

Large nucleus—most of the cell is taken up by the nucleus (a high nuclear to cytoplasmic ratio). Immature chromatin—the nuclear chromatin looks as if it composed of fine dots. One can visualize this chromatin as many tiny points made by the tip of a sharp pencil on a piece of paper. Monocyte chromatin is more linear and dark, looking like smudged pencil lines. Lymphocyte chromatin looks to be colored in heavy crayon. Prominent nucleolus.
Conformable nuclear membrane—the nuclear membrane often conforms to the shape of the cytoplasmic membrane. The nucleus appears squishy. A lymphocyte nucleus appears rigid. Scant cytoplasm. Few to no cytoplasmic granules-blasts usually lack the numerous granules seen in mature granulocytes. Occasional granules may be present. Acute promyelocytic leukemia is an exception. Auer rods-orange-pink, needle-like cytoplasmic structures in blasts of myeloid lineage. These may be numerous in acute promyelocytic leukemia.

In some forms, the transgenic zebrafish may show no changes in mature myelomonocytic cells.

The transgenic zebrafish may have increased proportion of R3 (lymphoid and HPSC) and R4 (precursor) population as seen by flow cytometry.

The kidney marrow (KM) cells may exhibit features consistent with cell proliferation. In some embodiments, the leukemic clones containing kidney marrow cells are successfully grafted into irradiated wild-type zebrafish as determined by morphological and flow cytometry assessments. For instance, in the Examples, kidney marrow from the Tg(Runx1:FLT3^{ITD}IDH2^{R172K}) and Tg(Runx1:FLT3^{ITD}IDH2^{R172K}) zebrafish are transplanted to lethally irradiated wildtype zebrafish, resulting in shorter survival. Successful engraftment of leukemic clones is demonstrated by increased blasts in the blood and kidney marrow, increased cellularity of the kidney marrow 30 days post transplantation, increased expression of human FLT3^{ITD} and IDH2 in the recipient's kidney marrow and increased macrophages and blasts in the recipient's spleen.

### 3. Molecular Characteristics

The disclosed transgenic zebrafish may demonstrate development of specific hematopathological features consistent with AML observed in mammals and humans. At the embryonic level, the transgenic zebrafish may express markers associated with definitive hematopoietic stem cells, primitive neutrophils, and pan-leukocytes. Exemplary markers of definitive hematopoietic stem cells include cardiac MYB proto-oncogene (cmyb), runx family transcription factor 1 (runx1). Exemplary markers of primitive neutrophils include myeloperoxidase (mpo) and Sudan black B (SBB). Exemplary pan-leukocyte markers include I-plastin.

The disclosed transgenic zebrafish may express features of effector cells consistent with human AML. The transgenic zebrafish may express increased markers of T-cell progenitors and common lymphoid progenitors including rag1 and cmyb. The transgenic zebrafish may express decreased markers of B-cell development including cd79a, pax5, and cd9a.

In some forms, the transgenic zebrafish exhibit gene expression patterns consistent with mammalian AML models. In an exemplary embodiment, the Tg(Runx1:FLT3^{ITD}IDH2^{R140Q}) and Tg(Runx1:FLT3^{ITD}IDH2^{R172K}) zebrafish lines demonstrate increases in genes associated with common lymphoid progenitor (CLP) cells, macrophages, hematopoietic stem and progenitor cells (HPSC-MPP), common myeloid progenitor (CMP) cells, thrombocytes, erythroid progenitors, B-cell and hematopoietic stem cell (HSC) populations and decrease in erythroid populations. In a second exemplary embodiment, the Tg(Runx1:FLT3^{ITD}IDH2^{R140Q}) zebrafish line demonstrate higher up-regulated genes in the CLP, HSPC-MPP, GMP and stromal cells, and more down-regulated genes in the HSPC-MPP, CMP, GMP, myeloid progenitors and myelocytes consistent with mammalian models of AML.

### III. Methods of Making Transgenic Zebrafish

The disclosed methods of generating the transgenic zebrafish includes (1) preparing entry clones containing gene sequences each encoding human AML-associated proteins of interest, zebrafish tissue- or cell-type specific promoters or reporter proteins, (2) incorporating the entry clones into a destination vector tagged with a transgenesis marker, (3) co-injecting the transgene constructs and the mRNA encoding the Tol2 transposase into zebrafish embryos, (4) phenotypic analysis and generation of stable F1 clones, and (5) crossing the F1 clones to produce heterozygous F2 double transgenic clones.

### A. Gene/Genome Editing

Methods of producing transgenic zebrafish lines using site-directed mutagenesis are well known in the art. Any number of transgenic tools may be optimized and used to prepare the transgenic zebrafish as disclosed. These include, but are not limited to Tol2-mediated transgenesis, cre-mediated site-specific recombination, I-Sce1 meganuclease transgenesis and BAC-mediated transgenesis. Preferably, the Tol2 transposition system is used to generate the transgenic zebrafish as disclosed. In the Tol2 system, the construct for introducing the DNA into the zebrafish genome typically consists of two minimal cis regulatory sequences from the Tol2 element positioned at the 5′ and the 3′ ends of a minimal promoter followed by a fluorescent reporter protein. The Tol2 containing vector can accommodate DNA inserts of up to 11 kb. Such DNA constructs are injected together with the mRNA encoding for a transposase gene directly into the cells of the one-stage embryos. The transgene of interest will be inserted into the zebrafish genome randomly via the transposon system. These embryos are raised, and the adult fish are screened to identify the founders, in which the insertions were created in the germline that can be transmitted to the next (F1) generation. In some embodiments, transgenic tools for conditional mutagenesis and site-specific recombination are used to produce the disclosed transgenic zebrafish. In these forms, conditional gene expression systems permit stable expression of human AML-associated genes in a cell type-specific and temporally restricted manner. Examples of conditional transgenic tools include but are not limited to the GAL4-UAS system, CRISPR/Cas9-based gene editing, Zinc finger nucleases, morpholinos, TALEN-mediated gene editing, mifepristone-inducible LexPR systems, Cre/loxP and FLP/FRT site specific recombination systems, and any combination thereof. Such transgenic tools are well known to those of skill in the art.

### 1. Cloning of Wildtype (WT) and Mutant Genes

Wildtype (WT) and mutant forms of human AML-associated genes are obtained from various cell lines for cloning. FLT3-ITD from the pMSCV-neo-FLT3^{ITD} plasmid, NPM1 type A mutation from the Cellosaurus OCI-AML-3 cell line, IDH2^{R140Q}, IDH2^{R172K} and DNMT3A^{R882H} from KG-1 cell line or AML patients via site-directed mutagenesis of corresponding WT genes. In some embodiments, CRISPR/Cas9 is used to generate mutant zebrafish that have knock-out of one or more of the genes listed in Table 1. In some embodiments, one or more of the mutations listed in Table 1 were cloned from patient samples. Preferably, the Tol2 plasmids containing wild-type and mutant genes are created based on multisite Gateway cloning using a BP reaction and a LR reaction. Gateway cloning is well known to those of skill in the art. Other cloning methods known in the art may also be used for gene cloning including Gibson Assembly, Golden Gate Assembly, Restriction Cloning and TOPO (TA) Cloning methods.

In the first step, Gateway cloning is used to produce entry clones. For the disclosed zebrafish lines, the mutant forms of human AML-associated genes are cloned into a pDONR221 donor vector to produce a “middle” clone (pME-IDH2^{R140Q} or IDH2^{R172K}) using a Gateway BP reaction. In the Gateway BP reaction, the human-AML gene of interest is amplified by PCR using an attB tagged primer pair and the PCR-purified products are incorporated into the pDONR221 donor vector that includes the attP sites to form an entry clone. The vector mixture is incubated with BP Clonase II overnight at 25° C. The resulting entry clone contains the human-AML gene of interest flanked by the attL sites. To construct the complete Tol2 plasmid, p5E, p3E and pDest plasmid vectors may be used. Exemplary entry clones are provided in the Examples and include p5E-Runx1+23 (Addgene #69602), pME-IDH2^{R140Q} or IDH2^{R172K}, and p3E-mCherrypA. Non-limiting examples of alternative markers to p3E-mCherrypA include p3E-IRES-EGFPpA, p3E-tag BFP, and p3E-RFP. Alternative donor vectors may be used to generate the entry clones including pDONRP4-PIR, pDONRP2R-P3, pDONOR201, pENTR/D-TOPO and pENTR11 donor vectors.

In the second step, the entry clones are incorporated into a destination vector to produce an expression clone of the final Tol2 integrable construct. The p5E-Runx1+23, pME-IDH2^{R140Q} or IDH2^{R172K}, and p3E-mCherrypA are integrated into the destination vector pDestTol2CG2 in a 1:1:1 ratio. The vector mixture is incubated with LR clonase overnight at about 25° C. Prior to competent cell transformation, the products of BP reaction and LR reaction are treated with proteinase K. Other destination vectors may be used to generate the expression clones including, but not limited to pDestTol2 pA, pDESTtol2pACrymCherry, pDESTtol2pACrymEgfp, pLenti CMV Puro DEST (w118-1), the doxycycline-inducible pLIX_402, pDONOR/Zeo.

In a third step, Tol2 transposase mRNA is generated by in vitro transcription using a pT3TS-Tol2 plasmid.

In a fourth step, successful recombination is confirmed by colony PCR and bi-directional DNA sequencing methods known to those of skill in the art.

### 2. Elements of Expression Sequences

a. Promoters

The expression sequences can include a promoter, enhancer, silencer, and necessary information processing sites, such as ribosome binding sites, RNA splice sites, poly adenylation sites and transcriptional terminator sequences. For example, the expression sequences may include an enhancer specific to human pluripotent stem cells (HPSC). Exemplary HPSC-specific enhancers include Runx1. Further, the expression sequences can include promoter sequences specific to neutrophil, macrophage, myeloid, erythroid and effector cells in blood circulation. Exemplary neutrophil-specific promoters include myeloperoxidase (mpo). Exemplary macrophage-specific promoters include macrophage-expressed gene 1 (mpeg1). Exemplary myeloid-specific promoters include lysozyme (lyz). Exemplary erythroid specific promoters include GATA-1 binding protein (gata1). Exemplary promoters specific to effector cells such as B-cell and T-cell lymphoid progenitors include recombination activation gene 2 (rag2). Exemplary leukocyte promoters include corola. Other inducible system includes UAS/gal4 which can be lineage specific, such as the GAL4/VP16-UAS system or Mifepristone-inducible LexPR system.

### B. Reporter Proteins

The transgenic zebrafish may express a reporter protein that is under the control of a specific expression sequence such as, but not limited to, the gata1 and corola promoter. Reporter proteins are useful for detecting or quantifying expression from expression sequences. For example, operatively linking nucleotide sequences encoding a reporter protein to a tissue specific expression sequence facilitates the study of lineage development. In some forms, the reporter protein serves as a marker for monitoring developmental processes. Many reporter proteins are known to one of skill in the art. These include, but are not limited to, β-galactosidase, luciferase, and alkaline phosphatase that produce specific detectable products. Fluorescent reporter proteins can also be used, such as green fluorescent protein (GFP), green reef coral fluorescent protein (G-RCFP), cyan fluorescent protein (CFP), red fluorescent protein (RFP or dsRed2), blue fluorescent protein (BFP) and yellow fluorescent protein (YFP). For example, by utilizing GFP, fluorescence is observed upon exposure to ultraviolet light or under a fluorescent microscope without the addition of a substrate. The use of reporter proteins that, like GFP, are directly detectable without requiring the addition of exogenous factors are preferred for detecting or assessing gene expression during zebrafish embryonic development. A transgenic zebrafish embryo, carrying a construct encoding a reporter protein and a tissue-specific expression sequence, such as an expression sequence that directs expression in KM cells provides a rapid, real time in vivo system for analyzing spatial and temporal expression patterns of proteins of interest in KM cells.

### B. Collection and Microinjection of Zebrafish Embryos

Methods of collecting and microinjecting plasmid DNA into zebrafish embryos are well-known to those of skill in the art. Described below are exemplary methods.

### 1. Collection of Zebrafish Embryos

The night prior to injection, wildtype Tubingen (TU) zebrafish are set in breeding tanks with dividers set in place. Total egg production is increased by grouping 2 females and 1 male. The dividers are removed the following morning and zebrafish are allowed approximately 20 minutes of undisturbed mating time. A strainer is used to collect the eggs from the breeding cages, rinsed with water, transferred to a petri dish with egg water and unfertilized eggs and debris are removed with a transfer pipette. Collected embryos are kept in E3 medium containing 0.29 g NaCl, 13.35 mg KCl, 48.4 mg CaCl₂·2H₂O, 51.5 mg MgCl₂·6H₂O in 1 liter of water (pH 7.2) at a temperature of 28.5° C. or 24.5° C.

The zebrafish are regrouped in larger tanks to produce additional rounds of eggs for injection. Timing of egg collection needs to be set appropriately to allow maximum egg production at the single cell stage.

An agarose chamber with multiple lanes is placed in the inverted lid of a petri dish and a transfer pipette is used to line the eggs into each lane. Excess egg water is removed from the slide.

### 2. Microinjection of Zebrafish Embryos

Microinjection methods are known to those of skill in the art. In an exemplary method, microinjection is performed with an injector such as a PLI-100A Pico-Injector (Harvard Apparatus, Massachusetts, USA), a micromanipulator (for example, a model MB-PSL, Kanetec Co, Japan) and a stereomicroscope (for example, a Nikon SMZ800, USA). Materials to be microinjected are diluted in an injection cocktail. A preferred injection cocktail contains 20 mM HEPES, 120 mM KCL, and 0.05% phenol red. To integrate the Tol2 transgene into the zebrafish genome, Tol2 plasmid (50-100 pg) is co-injected with Tol2 transposase mRNA (50-100 pg) into the cytoplasm of 1-cell stage embryo. An exemplary method includes (1) piercing the surface of the chorion and entering the cytoplasm in one smooth stroke while preventing puncturing or tearing of the yolk sac and (2) moving the injected eggs to a clean petri dish using a gentle stream of water.

### C. Screening for Transgenic Zebrafish

Methods of screening transgenic zebrafish are known in the art. In an exemplary method, the pDesTol2CG destination plasmid contains an EGFP or RFP fluorescent protein expressed under the control of the heart specific cmlc2 promoter to serve as a transgenesis marker. The cmlc2 promoter drives EGFP or RFP expression in cardiomyocytes in 2-day old F1 larvae, thereby allowing sorting of transgenic zebrafish using a fluorescence stereomicroscope such as an OLYMPUS MVX10.

### D. Genomic DNA Extraction and Genotyping of Transgenic Zebrafish

Methods of genomic DNA extraction and genotyping are well established in the art. In an exemplary method, adult zebrafish are anaesthetized with 300 mg/L tricaine methanesulfonate. The tail fins of the fishes are clipped and lysed in lysis buffer at 55° C. for 4 hours to overnight and heated to 98° C. for 10 minutes to inactivate the proteinase K to extract genomic DNA. A preferred lysis buffer cocktail contains 10 Mm Tris HCl (Ph 8.3), 50 Mm KCl, 0.3% Tween-20, 0.3% NP-40, and 1% proteinase K). PCR is performed to genotype the individual fish.

### E. Zebrafish Husbandry

Methods of breeding and storing zebrafish are known in the art. In an exemplary method, wildtype Tubingen (TU) and transgenic adult zebrafish are maintained and raised in aquariums at 28° C. and Ph 7-8 equipped with water filtration and ultraviolet sterilization. Zebrafish are housed in a standard light-dark (14 hours light/10 hours dark) cycle and fed 3 times daily with shrimp or ZM-300 fry food.

### IV. Methods of Using Transgenic Zebrafish

The disclosed transgenic zebrafish lines can be used in a variety of methods, such as, to examine the effects of mutated gene interactions and combinations thereof on pathology of human AML, both globally and in a tissue and cell-type specific manner. The disclosed transgenic zebrafish can be used to screen and evaluate more effective therapeutic agents for personalized treatment of human AML given a specific combination of mutated genes. Owing to their genetic conservation with humans, low maintenance costs, ability to be housed densely, and permeability to small molecules, it has previously been shown that transgenic zebrafish are able to serve as useful tools for drug screening for cancer therapeutics.

Accordingly, methods for identifying agents (e.g., small organic molecules) that can be used in the treatment of acute myelogenous leukemia are disclosed. These methods involve contacting embryos, larva or adult transgenic zebrafish disclosed with a candidate agent and analyzing the effect of the agent on an AML-related phenotype of the zebrafish. The effect of an agent can be assessed using an AML-related phenotypes such as survival, spleen size, decrease in myelopoiesis, restored erythropoiesis, whole kidney marrow cellularity, blasts and neutrophil counts, when compared to untreated controls. Myelopoiesis is defined as the development of non-lymphoid leukocytes. The AML-related phenotype can be accumulation of hematopoietic cells in the intermediate cell mass (ICM), loss of hematopoietic cell maturation as detected by analysis of a hematopoietic marker such as PU.1, GATA-1, myeloid-specific peroxidase (MPO), or SCL. In preferred examples, the methods involve the analysis of multiple zebrafish, which are present in separate wells of a multi-well plate and are contacted with different candidate agents. Further, in these examples, an automated system can advantageously be used to monitor the phenotypes of the zebrafish, as described elsewhere herein.

In some forms, the method includes contacting embryos, larva or adult fish with a test agent at test concentrations and test intervals to determine the therapeutic effect if any, of the test agent. A “test agent” as used herein, refers to any biologic or chemical entity whose therapeutic efficacy within the context of the specific combinations of mutations in AML disclosed herein, is unknown. Thus, the disclosed transgenic zebrafish or embryos containing the disclosed mutations can be used to investigate the therapeutic effect of a test agent on a transgenic zebrafish or embryo containing 2, 3, 4, 5, 6, 7, 8, 9, 10, 11, 12, or 13 of the following AML-associated gene mutations: FLT3^{ITD}, NPMc+, DNMT3A^{R882}, NRAS^{G12D}, IDH2^{R140Q}, IDH2^{R172K}, TET2^{-/-}, RUNX1^{-/-}, IDH1^{R132H}, WT1^{-/-}, TP53^{-/-}, SRSF2^{P95H}, ASXL^{-/-}, STAG2^{-/-}, and PHF6^{-/-}.

Small molecules can be directly added to the embryo growth medium and fluorescent transgenes make high throughput analysis of chemical effects possible.

As exemplified in the Examples, test agent at three concentrations (0.01 μM, 0.1 μM and 1 μM) is added to the embryos in E3 medium with (1-phenyl-2-thiourea) PTU at 6 hpf, and the medium changed every 24 hours. At 4 dpf, embryos are collected and fixed in 4% PFA for SSB staining. For adult fish, test agent (exemplified by Quizartinib and Enasidenib is diluted in a solution of H₂O, 10% DMSO (Sigma) and 0.01% Phenol Red (Sigma-Aldrich)) is diluted in suitable diluent and administered to the fish via oral gavage. Adult zebrafish can be anesthetized using 0.015% Tricaine. Once anesthetized, the zebrafish is propped vertically in a damp sponge, and 5 μl-10 μl of Quizartinib (10 mg/kg) or Enasidenib (100 mg/kg) is dispensed into the fish by the gavage apparatus. Treated zebrafish are then placed into an individual recovery tank with fresh sterile fish water immediately after the gavage procedure. The treatment regimen was repeated as desired for the particular test agent.

In another embodiment the disclosed leukemia transgenic lines can be crossed with reporter lines to generate lines with tissue and/or cell specific mutants, are then used for drug screening. In general, the disclosed transgenic lines are crossed with lines expressing a tissue or cell type specific promoter operably linked to a reporter protein, to obtain lines in which the disclosed combination of mutations are expressed in a cell or tissue specific manner dictated by the tissue or cell type specific promoter. Exemplary lines include, but are not limited to Tg(rag2:EGFP), Tg(mpo:EGFP), Tg(lyz:EGFP) and Tg(gata1: RFP). Tg(rag2:EGFP) and Tg(mpo:EGFP) zebrafish lines are commercially available. Tg(lyz:EGFP) and Tg(gata1: RFP) fish lines can be generated as disclosed in the Examples. For example the disclosed lines can be used to generate Tg(mpo:EGFP, gata1: RFP, Runx1:FLT3^{ITD}IDH2^{R172K/R140Q}) where mpo:EGFP in which mpo is the cell-type specific promoter, EGFP is the reporter protein; or Tg(lyz:EGFP, corola:DsRED, Runx1:FLT3^{ITD}IDH2^{R172K/R140Q}).

The present disclosure can be further understood by the following non-limiting examples and paragraphs.

1. A genetically modified zebrafish model of human acute myeloid leukemia (AML), wherein the zebrafish stably expresses genes comprising at least two human gene mutations selected from the group consisting of FLT3^{ITD}, NPMc+, DNMT3 A^{R882}, NRAS^{G12D}, IDH2^{R140Q}, IDH2^{R172K}, TET2^{-/-}, RUNX1^{-/-}, IDH1^{R132H}, WT1^{-/-}, TP53^{-/-}, SRSF2^{P95H}, ASXL^{-/-}, STAG2^{-/-}, and PHF6^{-/-}.
2. The zebrafish of paragraph 1, wherein zebrafish carries the specific mutation combinations FLT3^{ITD}, IDH2^{R140Q} and/or IDH2^{R172K}.
3. The zebrafish of paragraph 1 or 2, wherein zebrafish carries the specific mutation combinations: (a) FLT3^{ITD} and IDH2^{R140Q}, (b) FLT3^{ITD} and IDH2^{R172K}, (c) SRSF2^{P95H} NRAS^{G12D}; (d). asxl1^{+/-} IDH2^{R172K}; (e) FLT3^{ITD}SRSF2^{P95H}; or (f) asxl1^{+/-} SRSF2^{P95H}.
4. The genetically modified zebrafish of any one of paragraphs 1-3, further comprising a zebrafish specific expression sequence operably linked to a nucleic acid sequence encoding a fluorescent reporter polypeptide.
5. The genetically modified zebrafish of paragraph 4, wherein the fluorescent reporter polypeptide is selected from the group consisting of green fluorescent protein (GFP), green reef coral fluorescent protein (G-RCFP), cyan fluorescent protein (CFP), red fluorescent protein (RFP or dsRed2), blue fluorescent protein (BFP) and yellow fluorescent protein (YFP).
6. The genetically modified zebrafish of any one of paragraphs 1-5, wherein the zebrafish specific expression sequence is a neutrophil-specific promoter, macrophage-specific promoter, erythroid cell specific promoter, effector cell specific promoter or leukocyte promoter.
7. The genetically modified zebrafish of any one of paragraphs 1-6 comprising: mpo:EGFP, gata1:RFP, Runx1:FLT3^{ITD}IDH2^{R172K/R140Q} or lyz:EGFP, corola:DsRED, Runx1:FLT3^{ITD}IDH2^{R172K/R140Q}; or Runx1:FLT3^{ITD}SRSF2^{P95H}, Runx1:SRSF2^{P95H} NRAS^{G12D}.
8. The genetically modified zebrafish of any one of paragraphs 1-7, wherein expression of the expression product is transmitted through the germline.
9. The genetically modified zebrafish of any one of paragraphs 1-8, wherein the human zebrafish expression sequence and the nucleic acid sequence encoding the fluorescent reporter are contained in an exogenous construct.
10. The genetically modified zebrafish of any one of paragraphs 1-9, wherein the construct further comprises (a) intron sequences operably linked to the nucleic acid sequence encoding the fluorescent reporter, (b) a polyadenylation signal operably linked to the nucleic acid sequence encoding the fluorescent reporter, or both.
11. The genetically modified zebrafish of any one of paragraphs 1-10, wherein the genetically modified zebrafish is developed from, or is the progeny of a genetically modified zebrafish developed from, an embryonic cell into which the exogenous construct was introduced.
12. The zebrafish of any one of paragraphs 1-11, wherein zebrafish carries the specific mutation combinations: (a) FLT3^{ITD} and NPMc+, (b) FLT3^{ITD} and DNMT3A^{R882}, or (c) FLT3^{ITD}, NPMc+, and DNMT3A^{R882}.
13. The zebrafish of any one of paragraphs 1-11, wherein zebrafish carries the specific mutation combinations: (a) FLT3^{ITD} and NRAS^{G12D} (b) FLT3^{ITD} and IDH2^{R140Q}, or (c) FLT3^{ITD}, NRAS^{G12D} and IDH2^{R140Q}.
14. The zebrafish of any one of paragraphs 1-11 wherein zebrafish carries the specific mutation combinations: FLT3^{ITD}, NPMc+, DNMT3A^{R882}, NRAS^{G12D}, IDH2^{R140Q}, IDH2^{R172K}, TET2^{-/-}, RUNX1^{-/-}, IDH1^{R132H}, WT1^{-/-}, TP53^{-/-}, SRSF2^{P95H}, ASXL^{-/-}, STAG2^{-/-}, and PHF6^{-/-}.
15. A method of using the genetically modified zebrafish of any one of paragraphs 1-14 for drug screening comprising contacting the genetically modified zebrafish with a test agent, at test concentrations and test intervals to determine the therapeutic effect if any, of the test agent.
16. The method of paragraph 15, wherein the zebrafish are embryos, larvae, juveniles or adults.
17. A method of identifying a candidate therapeutic compound for the treatment of AML, the method comprising: contacting the genetically modified zebrafish of any one of paragraphs 1-14 with a test compound and determining the effect of the agent on a phenotype selected from the group consisting of survival, spleen size, decrease in myelopoiesis, erythropoiesis, whole kidney marrow cellularity, blasts and neutrophil counts.
18. The method of paragraph 17, wherein the genetically modified zebrafish is an embryo.
19. The method of paragraph 17, wherein the genetically modified zebrafish is an adult fish.
20. The method of paragraph 17, wherein the genetically modified zebrafish is at the larval stage.
21. The method of any one of paragraphs 17-20, wherein detection of an improvement in the phenotype indicates identification of an agent that can be used in the treatment of AML.

### EXAMPLES

### Example 1

### Methods

### Zebrafish Husbandry

Wildtype Tubingen (TU), Tg(rag2: EGFP) and Tg(mpo: EGFP) zebrafish lines were purchased from Zebrafish International Resource Center (ZIRC, USA). Tg(lyz: EGFP) and Tg(gata: RFP) fish lines were generated by microinjection of the respective tol2 construct with tol2 transposase into the TU embryos at one-cell stage. F1 reporter lines were screened and identified by fluorescence microscope based on EGFP or RFP fluorescent signals. All the fish lines were maintained and raised at 28° C. in 14 hours light-10 hours dark cycle in an automatically circulating system, Embryos were kept in E3 medium (5 mM NaCl, 0.17 mM KCl, 0.33 mM CaCl₂), 0.33 mM MgSO₄) and staged as previously described (Kimmel et al, 1995). The study was approved by the Committee of the Use of Laboratory and Research Animals (CULATR) in the University of Hong Kong (HKU).

### Generation of Transgenic Zebrafish Lines

Tol2-mediated transgenesis system was used to generate single transgenic fish line Tg(Runx1: IDH2^{R140Q}) and Tg(Runx1: IDH2^{R172K}) in which human IDH2^{R140Q} or IDH2^{R172K} were expressed under the control of the HSPC-specific Runx1 enhancer [Tamplin, O. J., et al. Cell, 2015.]. Briefly, human sequence of IDH2^{R140Q} or IDH2^{R172K} was cloned into the pDONR221 vector to generate the “middle” clone (pME-IDH2^{R140Q} or IDH2^{R172K}) by Gateway BP reaction. The final Tol2 integrable construct was generated via multisite Gateway LR reaction in which three entry clones [p5E-Runx1+23 (Addgene #69602), pME-IDH2^{R140Q} or IDH2^{R172K}, and p3E-mCherrypA] were incorporated into the destination vector pDestTol2CG with EGFP fluorescent protein expressed under the control of the heart-specific cmlc2 promote which was served as a transgenesis marker. Each single transgenic line was generated by co-injecting 50 pg-100 pg of the respective Tol2 construct and Tol2 transposase mRNA into the wildtype TU zebrafish embryos at the one-cell stage. Founders were identified by PCR and EGFP fluorescence of the heart. F1 single transgenic fish were generated by outcrossing the identified founder fish with wildtype TU fish. Flembryos with positive heart EGFP fluorescence at 2dpf were raised to adulthood, and the genotype of F1 fish were further confirmed by PCR of IDH2 using gDNA from fin clip. Generation of the Tg(Runx1:FLT3^{ITD}) transgenic line was described previously [He et al., EMBO 2020]. Double transgenic fish Tg(Runx1:FLT3^{ITD} IDH2^{R140Q}) and Tg(Runx1:FLT3^{ITD}IDH2^{R172K}) were generated by crossing the single transgenic lines of Tg(Runx1:IDH2^{R140Q/R172K}) with Tg(Runx1:FLT3^{ITD}), and were identified by PCR of FLT3 and heart EGFP fluorescence screening of IDH2. The expression of FLT3 and IDH2 mRNA in F1 adult fish were detected by q-PCR. F1 transgenic fish were also outcrossed with different transgenic reporter lines, including Tg(rag2:EGFP), Tg(mpo: EGFP), Tg(lyz: EGFP) and Tg(gata1: RFP).

### Whole Mount In Situ Hybridization (WISH) Assay

Whole mount in situ hybridization (WISH) was performed as described previously (He et al, 2014; Ma et al, 2017). Zebrafish embryos collected at 12hpf, 24hpf, 36hpf, 48hpf, 3dpf and 5dpf were fixed in 4% paraformaldehyde (PFA) at 4° C. overnight, followed by stepwise dehydration with methanol of increasing concentration, and were incubated at −20° C. overnight for in situ hybridization. WISH was performed using probes for zebrafish cmyb, pu.1, l-plastin, rag1, runx1, mpo and Imo2. Briefly, on day 1, pre-fixed and permeabilized embryos were rehydrated, digested (embryos beyond 24 hpf) by proteinase K, re-fixed with 4% PFA, pre-hybridized in PHB− buffer at 65° C.° C. for 6 h, and hybridized in PHB+ buffer with DIG-labeled WISH probes (0.5 ng/μl) at 65° C. overnight. After serial washing on day 2, the embryos were incubated with AP-conjugated anti-DIG antibody (1:5,000 in PBST with 5% lamb serum) at 4° C. overnight with constant shaking. After serial washing on day 3, gene expression signals were developed by NBT/BCIP substrate (Roche) in one to six hours at room temperature or overnight at 4° C. Then embryos were mounted in 3% methylcellulose and imaged using a Nikon SMZ800 fitted with a CWHC-1080B 4K cMOS camera (Chinetek Scientific). The images were analyzed using ImageJ. WISH was performed in at least three biological triplicates. Thirty embryos were included in each experiment.

### Sudan Black B (SBB) Staining

Zebrafish embryos were collected and fixed in 4% PFA overnight at 4° C., followed by repeated washing with PBST. They were then incubated in Sudan Black B Staining Reagent diluted 1:10 in 70% ethanol for 30 minutes at room temperature. Afterwards, embryos were washed extensively in 70% ethanol, then serially rehydrated with decreasing ethanol concentration in PBST, eventually in PBST only. Embryos were then mounted in 3% methylcellulose and imaged with CWHC-1080B 4K cMOS camera (Chinetek Scientific) attached to a Nikon SMZ800 stereomicroscope and analyzed using ImageJ.

### Quantitative Real-Time PCR

mRNA was extracted from the whole kidney marrow (WKM) of the transgenic fish or wildtype TU fish with Trizol (Invitrogen) using the RNA microprep kit (R2040 Zymo, USA). cDNA was generated using HiScript II (Vazyme) according to the manufacturer's instruction. Real time quantitative PCR was performed using SYBR Green reagents and StepOnePlus Real-Time PCR System (Applied Biosystems). The sequences of PCR primers were zebrafish 8-Actin forward (AATGAGCGTTTCCGTTGCC) (SEQ ID NO:1) and reverse (CAGGTCCTTACGGATGTCCAC) (SEQ ID NO:2), IDH2 forward (CCAAACCGTGACCAGACTGA) (SEQ ID NO:3) and reverse (CTCATCAGGGGTGATGGTGG) (SEQ ID NO:4), and FLT3 forward (TACAAATCAAGATCTGCCTGTGA) (SEQ ID NO:5) and reverse (CTGAGCTCTGGGGTCTCAAC) (SEQ ID NO:6). Fold change of gene of interest was calculated using the 2 (−ΔΔCt) method.

### Whole Kidney Marrow (WKM) Collection for Flow Cytometry and Cytospin Analysis

Zebrafish WKM were harvested from two to twelve months old transgenic or wildtype TU fish. The collected KM was dissociated in 0.9×PBS with 5% fetal bovine serum (FBS) by pipetting and filtered through a 40 μm nylon cell strainer (Corning, NY, USA). The cells were then centrifuged at 500 g for 5 minutes, resuspended, filtered, and washed with 0.9×PBS with 5% (FBS) at least twice. Flow cytometry of the KM was performed using a MoFlo XDP flow cytometer (Beckman coulter). The data was analyzed using FlowJo™ v10.6.1 software. Cytospin preparations were made with 2×10⁵ to 3×10⁵ cells cytocentrifuged at 400 rpm for 5 minutes onto microscope glass slides (Thermo scientific). The glass slides were dried at room temperature for more than 12 hours, stained with Wright-Giemsa stain, and imaged with Nikon DS-Ri2 digital camera fitted to a Nikon Eclipse Ni-U microscope.

### Blood Collection and Blood Smear

The blood collection method and apparatus were adapted from Zang, Liqing et al. JoVE, 2015. Glass capillary needles (GC100TF-15, Warner Instruments, USA) were soaked in 5 mg/ml heparin and air-dried at room temperature at least 1 hour. Fish were anesthetized with 0.015% Tricaine in E3 medium and placed onto a dissection board covered with wet paper towels. The blood collection apparatus was made of a heparinized needle, a silicone tubing (0.8 mm ID, Biorad) and a distritip microsyringe (125 μl, Gilson). Blood was collected by inserting the glass needle at a 45290° angle into the dorsal aorta of the adult fish and 5-10 μl of blood was drawn by gently moving the syringe up. The collected blood was subsequently dropped onto a glass slide and the blood smear procedure was performed right after. The glass slides were dried at room temperature for more than 12 hours, stained with Wright-Giemsa stain, and imaged with Nikon DS-Ri2 digital camera fitted to a Nikon Eclipse Ni-U microscope.

### In Vivo Microscopy

Adult fish with different reporter background, including Tg(mpo: EGFP), Tg(lyz: EGFP), Tg(rag2: EGFP) and Tg(gata1: RFP) were anesthetized with 0.015% Tricaine in E3 medium and placed into a petri dish, whole animal images were taken with a Carl Zeiss AxioZoom.V16 microscope and Axiocam 208 color camera. Images were captured using ZEN 3.2 Blue Edition and analyzed using ImageJ.

### Zebrafish Whole Kidney Marrow Transplantation

WT recipient fish were irradiated at 25Gy two days prior to the transplantation. On the day of the transplantation, WKM cells were collected in 0.9×PBS with 5% FBS from the double transgenic Tg(Runx1: FLT3^{ITD}IDH2^{R140Q/R172K}) and wildtype TU donor fish. Cells were then resuspended in injection medium with 0.9×PBS, 5% FBS, 0.5 U/μL heparin and 0.2 U/μL Dnase I to give a final concentration of 2×10⁵ cells/μL. Each recipient fish was anesthetized using 0.015% Tricaine and placed onto a sponge under the dissection microscope. A total of 5×10⁵ cells were transplanted into the cardiac chamber of the recipient by using a capillary glass needle (GC100TF-15, Warner Instruments, USA). After injection, the injected fish were immediately returned to sterilized fish water and fed with standard shrimp meals. The survival of the transplanted fish was monitored and recorded daily.

### Drug Treatment on Embryos and Adult Fish

Tg(Runx1:FLT3^{ITD}IDH2^{R140Q/R172K}) double transgenic and WT zebrafish embryos were treated with either FLT3 inhibitors Gilteritinib (Selleck S7754), Quizartinib (MedChemExpress HY-13001), Midostaurin (Selleck S8064), or IDH2 inhibitor Enasidenib (MedChemExpress HY-18690). Drugs at three concentrations (0.01 μM, 0.1 μM and 1 μM) were added to the embryos in E3 medium with (1-phenyl-2-thiourea) PTU at 6 hpf, and the medium was changed every 24 hours. At 4 dpf, embryos were collected and fixed in 4% PFA for SSB staining. For adult fish, Quizartinib and Enasidenib were diluted in a solution consisted of H₂O, 10% DMSO (Sigma) and 0.01% Phenol Red (Sigma-Aldrich) and administered to the fish via oral gavage. Adult zebrafish were anesthetized using 0.015% Tricaine. Once anesthetized, the zebrafish was propped vertically in a damp sponge, and 5 μl-10 μl of Quizartinib (10 mg/kg) or Enasidenib (100 mg/kg) was dispensed into the fish by the gavage apparatus. Treated zebrafish were then placed into an individual recovery tank with fresh sterile fish water immediately after the gavage procedure. The drug regimen was repeated daily for 14 days. The gavage apparatus was consisted of a 10 μl microliter syringe (Gaoge, Shanghai, China) and a polyethylene tubing (BTPE-50, INTECH, USA), and was adapted from Collymore et al., 2013 and Dang, Michelle, et al., 2016.

### Single Cell RNA Sequencing

Single viable cells were collected from kidney marrows from 7 months old transgenic fish and WT fish into 0.9×PBS with 5% FBS as described above. The cells were examined by microscope after 0.4% Trypan blue coloring. The single-cell library was constructed using the Chromium™ Controller and Chromium™ Next GEM Single Cell 3′ Kit v3.1 (10× Genomics, Pleasanton, CA). Briefly, single cells, reagents and Gel Beads containing barcoded oligonucleotides were encapsulated into nanoliter-sized GEMs (Gel Bead in emulsion) using the GemCode Technology. Lysis and barcoded reverse transcription of polyadenylated mRNA from single cells were performed inside each GEM. Post RT-GEMs were cleaned up and cDNA were amplified. cDNA was fragmented and fragments end were repaired, as well A-tailing was added to the 3′ end. The adaptors were ligated to fragments which were double sided SPRI selected. Another double sided SPRI selecting was carried out after sample index PCR. The final library was quality and quantitated in two methods: check the distribution of the fragments size using the Agilent 2100 bioanalyzer and quantify the library using real-time quantitative PCR (QPCR) (TaqMan Probe). The final products were sequenced using the DNBSeq™ platform (BGI-HK, China).

### Statistical Analysis

Data were analyzed using Prism9 software. The data were expressed as mean+standard error of the mean (SEM). Student t-test was used for pairwise comparison. The level of significance was indicated as *P<0.5, **P<0.01, ***P<0.001.

### Results

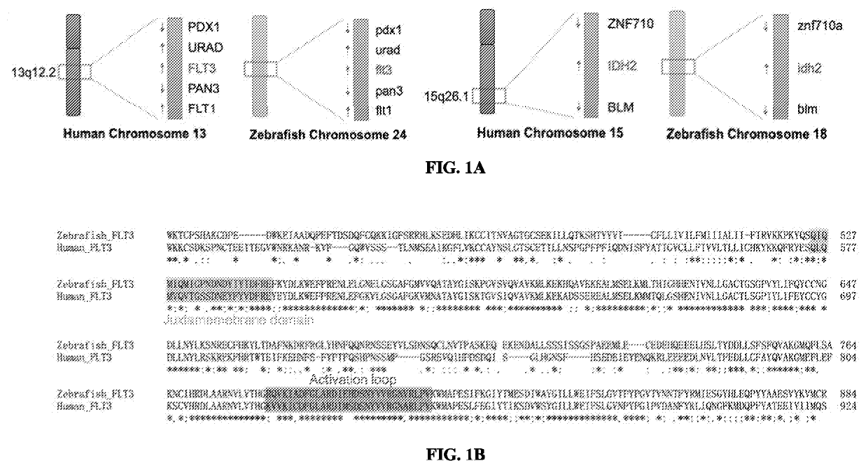

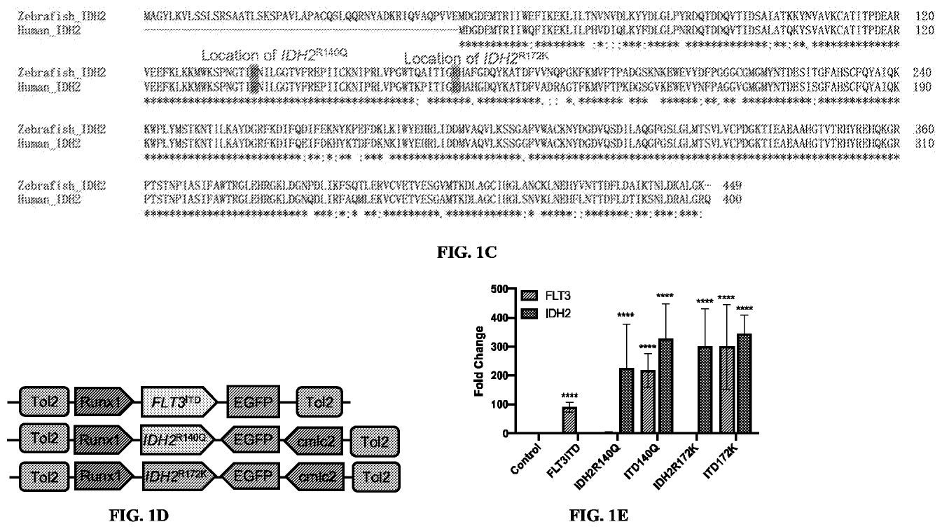

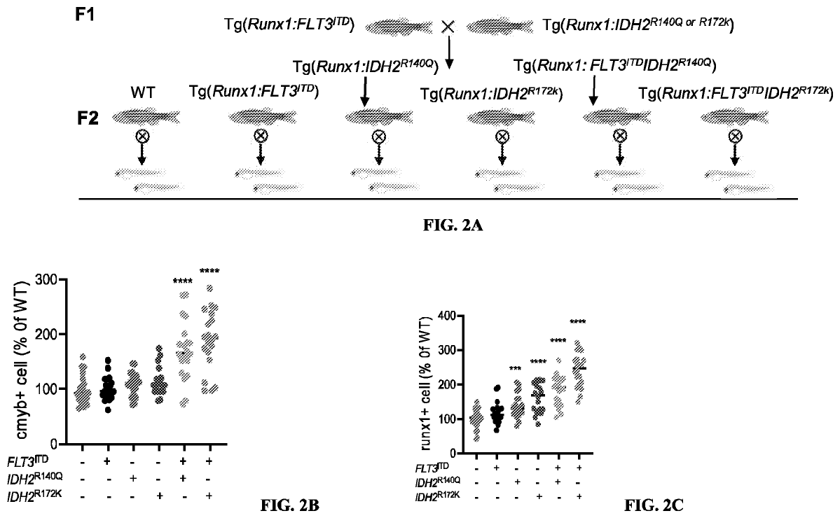

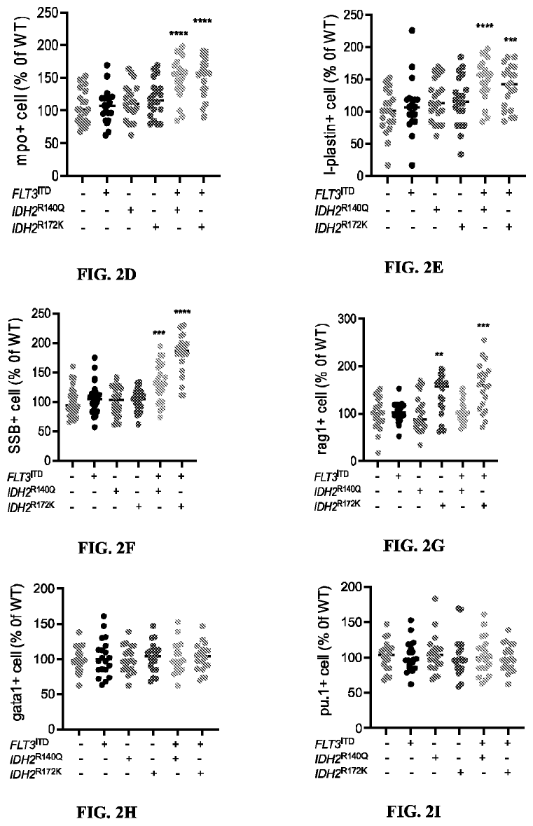

Stable transgenic lines expressing Tg(Runx1:FLT3^{ITD}) and Tg(Runx1:IDH2^{R140Q}) or Tg(Runx1: IDH2^{R172K}) Zebrafish flt3 and idh2 showed remarkable similarities in amino acid sequence and syntenic neighboring genes (SNGs) to those of human (FIG. 1-C), suggesting orthologous relationships. Transgenic Tg(Runx1:FLT3^{ITD}), Tg(Runx1:IDH2^{R140Q}) and Tg(Runx1: IDH2^{R172K}) were generated using Tol2 transgenesis (FIG. 1D). Successful integration of the Tol2 construct was shown by cmlc2:EGFP expression in the developing heart by measuring cardiac EGFP fluorescence signal in embryos at 24hpf and 48hpf. Adult zebrafish was outcrossed with wildtype (TU) to confirm germline transmission. Expression of mutant genes in F1 WKM was confirmed. Specifically, FLT3 and IDH2 mutant fish were confirmed by PCR genotyping of FLT3 and IDH2 using genomic DNA from fin clip of WT siblings and transgenic zebrafish (F1) at 2 months old. (FIG. 1E). F1 Tg(Runx1:FLT3^{ITD}) was crossed with Tg(Runx1: IDH2^{R140Q}) or Tg(Runx1:IDH2^{R172K}) (FIG. 2A). Hematopoietic gene expression in F2 embryos was examined by WISH and genotyped individually afterwards. Embryos co-expressing FLT3^{ITD} and IDH2^{R140Q} or IDH2^{R172K} showed significant increase in markers associated with definitive hematopoietic stem cells (cmyb, runx1; FIG. 2B and FIG. 2C respectively); primitive neutrophils (mpo, SBB; FIG. 2D and FIG. 2F respectively) and pan-leukocyte marker I-plastin (FIG. 2E). Intriguingly, only embryos carrying IDH2^{R172K} but not IDH2^{R140Q} showed increase in T-cell marker (rag1; FIG. 2G) in the developing thymus. Genes associated with primitive erythropoiesis (gata1; FIG. 2H) and early myeloid progenitor (pu.1; FIG. 2I) were unaffected by this oncogene expression.

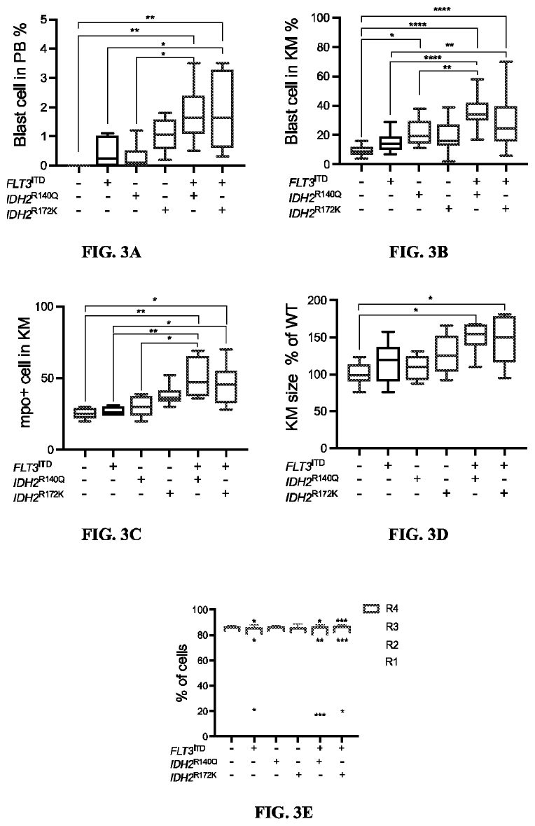

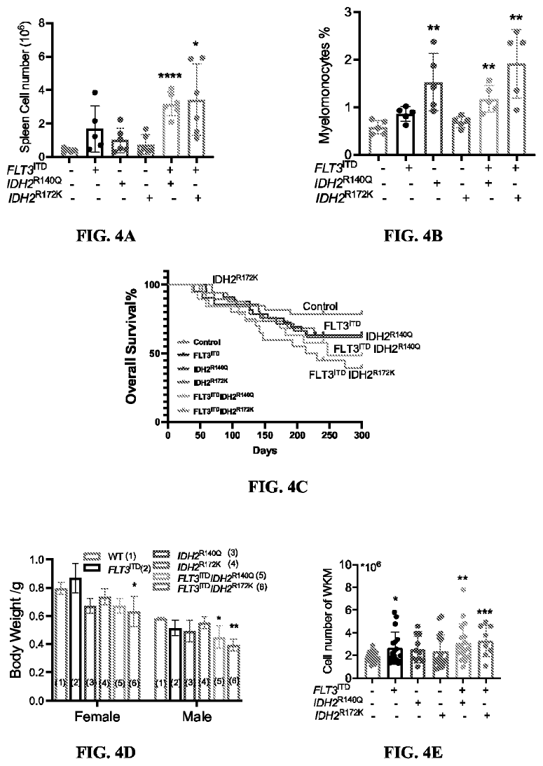

F2 larvae were raised to adulthood and their hematopathologic features were examined. Compared with their wildtype and single transgenic siblings, Tg(Runx1:FLT3^{ITD}IDH2^{R140Q}) and Tg(Runx1:FLT3^{ITD}IDH2^{R172K}) fish showed increases in circulating blasts (FIG. 3A, 3B), KM cellularity (FIG. 4E) and MPO+ myeloblasts (FIG. 3C), as early as 6 months. They were crossed to Tg(mpo:EGFP) and the EGFP signals were used to define the KM size. Both double transgenic fish showed significant increase in KM size (FIG. 3D). When evaluated by flow cytometry, they showed significant decrease in prevalence of R1, denoting erythropoiesis. There were significant increases in prevalence of R4, denoting myeloid precursors or erythroid precursors, and R3, which in wildtype adult fish represented predominantly lymphocytes and in double transgenic fish represented myeloblasts and lymphocytes. There was no significant change in R2, representing mature myelomonocytic cells (FIG. 3E). Their spleen also showed significant increase in size and cellularity with an increase in number of macrophage and myeloid and increase in proportion of R2 and R4 population in flowcytometry at 6-12 months of age (FIGS. 4A-D). After 100 days of age, they began to lose weight and their survival became shortened compared with their wildtype siblings (FIGS. 4A-D).

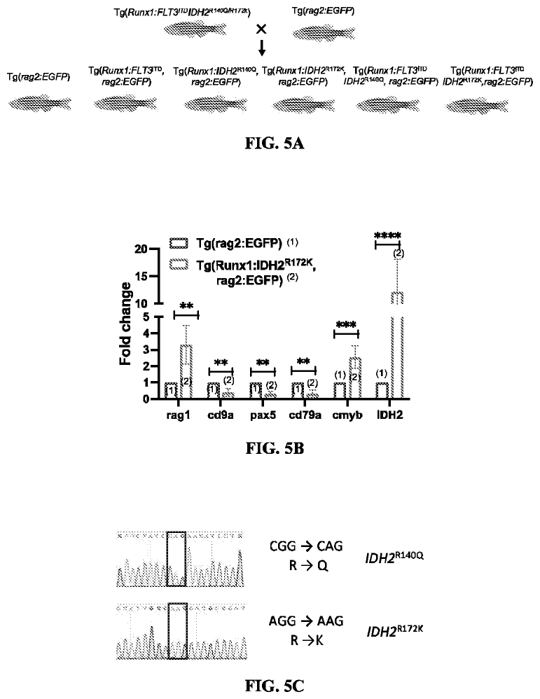

The T-cell development in adult fish was then examined given the increase in rag1 expression in F2 embryos carrying Tg(Runx1: IDH2^{R172K}) and Tg(Runx1:FLT3^{ITD}IDH2^{R172K}). These embryos were raised to adulthood and crossed with Tg(rag2:EGFP) fish in which T-cells in the thymus showed GFP (FIG. 5A). The thymus from mutant and WT zebrafish was visualized via fluorescent microscopy. The thymus of adult transgenic Tg(rag2:EGFP/Runx1: IDH2^{R172K}) and Tg(rag2:EGFP/Runx1:FLT3^{ITD}IDH2^{R172K}) fish were larger than the IDH2^{R1410Q} counterparts. The enlarged thymus expressed mutated human and showed increase in rag1 and cmyb (T-cell progenitors and common lymphoid progenitors) but decrease in cd79a, pax5 and cd9a (B-cell) expression (FIG. 5B). The presence of IDH2 hotspot mutations in the WKM and thymus of the mutant fish was confirmed via sanger sequencing (FIG. 5C).

### Transplantability of Leukemic Cells

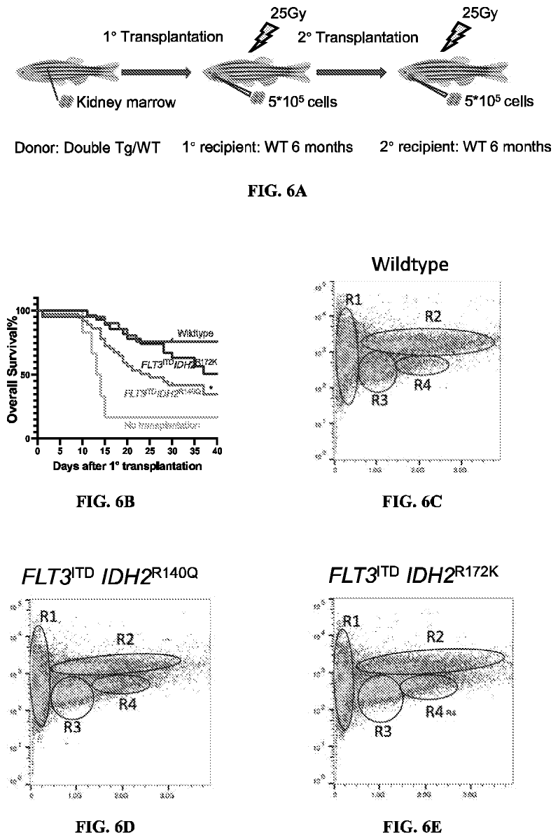

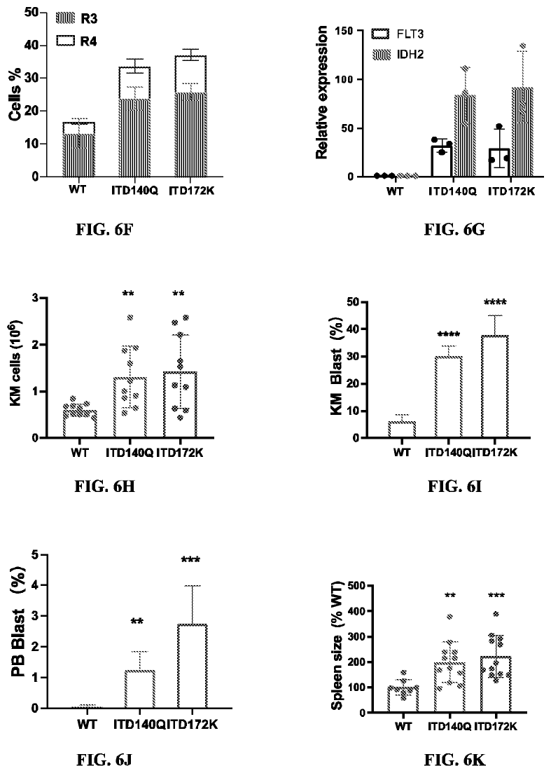

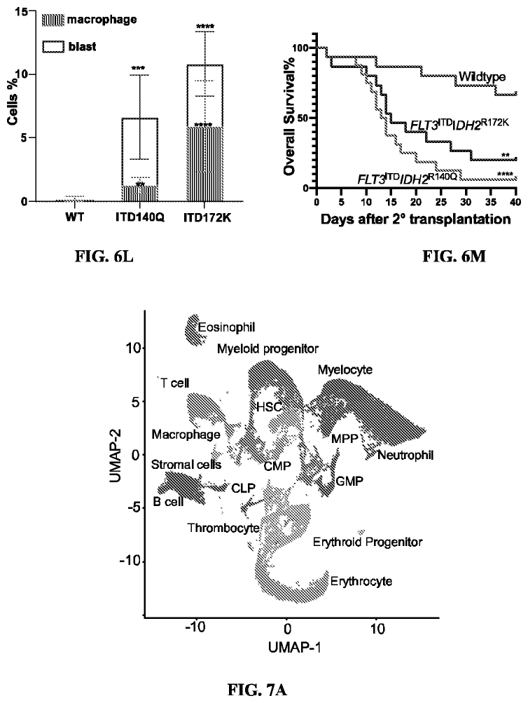

The ability of Tg(Runx1:FLT3^{ITD}IDH2^{R140Q}) and Tg(Runx1:FLT3^{ITD}IDH2^{R172K}) KM to propagate upon transplantation was examined (FIG. 6A). Lethally irradiated wildtype adult fish could be rescued by wildtype kidney marrow. Tg(Runx1:FLT3^{ITD}IDH2^{R140Q}) and Tg(Runx1:FLT3^{ITD}IDH2^{R172K}) recipients showed a significantly shorter survival compared with those who received wildtype marrow (FIG. 6B). Successful engraftment by leukemic clones was shown by increase in blasts in blood and KM and increase in cellularity of the latter on 30 days post transplantation, both by morphologic and flow cytometric assessment (FIGS. 6C-F). The recipient marrow showed expression of human FLT3^{ITD} and IDH2, which were not present in the recipients of wildtype marrow (FIG. 6G). Spleen size in the recipients has increased and there were also increases in macrophage and blasts in the spleen of recipients transplanted with FLT3^{ITD}IDH2^{R172K} and FLT3^{ITD}IDH2^{R140Q} kidney marrow (FIGS. 6H-L). Secondary transplantation was also performed. Leukemic engraftment was more aggressive and 80% of the Tg(Runx1:FLT3^{ITD}IDH2^{R140Q}) secondary recipients died within 20 days post KM transplantation and 80% Tg(Runx1:FLT3^{ITD}IDH2^{R172K}) secondary recipients died within 30 days post KM transplantation (FIG. 6M).

### Single Cell Transcriptome Analysis

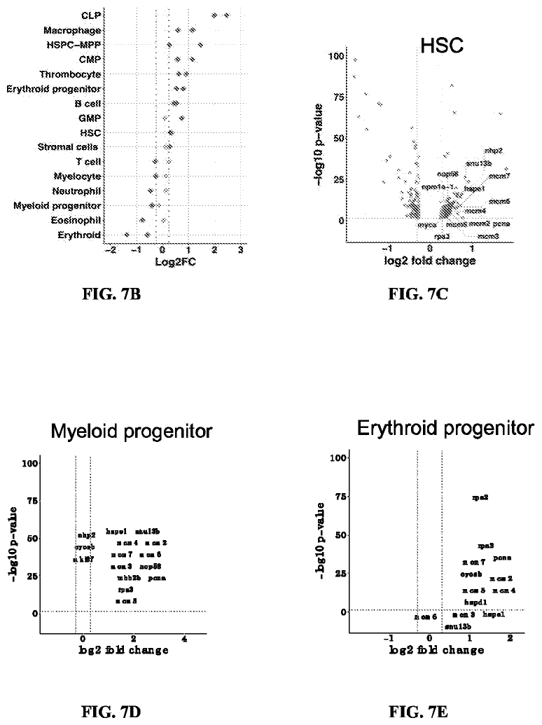

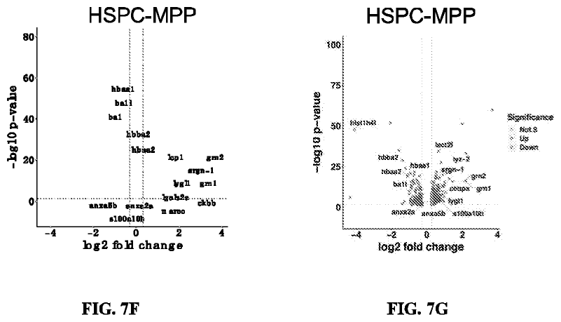

Single cell transcriptome analysis was performed in 9 fish [WT=3, Tg(Runx1:FLT3^{ITD}IDH2^{R140Q})=3, Tg(Runx1:FLT3^{ITD}IDH2^{R172K})=3]. After removal of doublets and filtering by sequencing QC, 34924 cells were included in subsequent analyses. Unsupervised clustering showed 16 clusters based on reported lineage-specific gene signatures using the Uniform Manifold Approximation and Projection (UMAP) in the R Seurat Package (FIG. 7A). Single-cell RNA sequencing revealed enrichment of leukemic-associated gene signatures in FLT3^{ITD} and IDH2 double mutant zebrafish. Cell type classification based on key haematopoietic marker genes were identified in zebrafish, murine, and human. Compared with wildtype sibling fish, both transgenic fish showed increases in prevalence of CLP, macrophage, HSPC-MPP, CMP, thrombocyte, erythroid progenitor, B-cell and HSC populations but decrease in erythroid populations by proportion analysis (FIG. 7B). For example, markers associated with neutrophil cell populations had log 2 fold increase in expression including, mmp13a, mpx, lyz and cpab. Markers of erythroid cell populations showing log 2 fold increase in expression included ba1, hbaa2, his1h4l, and cpa5. Markers of myeloid progenitor cells showing log 2 fold increase in expression included s100a10b, grn1, anxa5b, runxlt, gata2b, and myb. Markers of myelocytes showing log 2 fold increase in expression included mpx, mmp13a, lyz, and cpa5. Differentially expressed genes in selected cell populations of the WKM of FLT3^{ITD} and IDH2 double mutant and WT zebrafish were also determined. FIGS. 7C-F are volcano plots showing the differentially expressed genes in HSC (FIG. 7C), myeloid progenitor (FIG. 7D), erythroid progenitor (FIG. 7E) and HSPC-MPP (FIG. 7F) from Tg(Runx1:FLT3^{ITD}IDH2^{R140Q}) and WT WKM. FIG. 7G is a volcano plot showing the differentially expressed genes in HSPC-MPP from Tg(Runx1:FLT3^{ITD}IDH2^{R172K}) and WT WKM. Genes with significant enrichment log 2 fold change of more than 0.25 or log 2 fold change of less than-0.25 are shown.

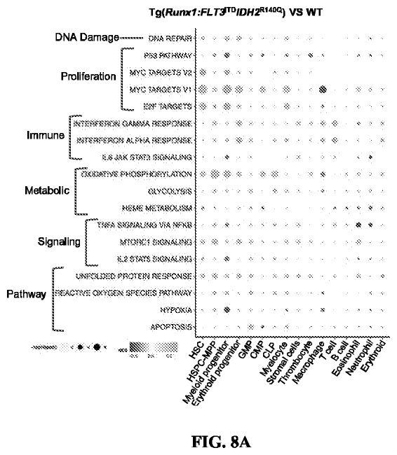

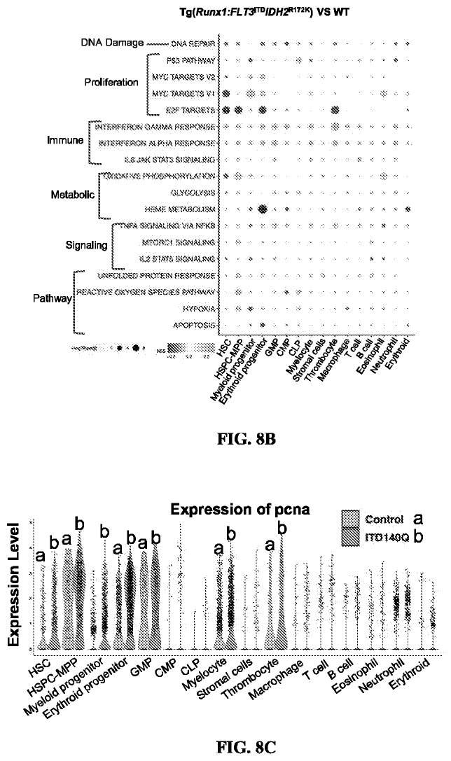

To further investigate the cooperative mechanisms between FLT3^{ITD} and IDH2 mutations, differentially expressed genes of each cell cluster from Tg(Runx1:FLT3^{ITD}IDH2^{R140Q/172K}) and WT were analyzed by the Molecular Signatures Database (MsigDB v7.4) hallmark gene set and Kyoto Encyclopedia of Genes and Genomes (KEGG) gene set enrichment analysis (GSEA) (FIG. 8A-FIG. 8B and data not shown). In Tg(Runx1:FLT3^{ITD}IDH2^{R140Q}) fish, HSC, HSPC-MPP, CMP, myeloid and erythrocyte progenitors and myelocytes showed significant enrichment of genes associated with cellular proliferation, oxidative phosphorylation and metabolism as well as MTORC1 signaling (FIG. 8A and FIG. 8C). Myeloid progenitor and CMP also showed negative enrichment of genes associated with cellular differentiation. Intriguingly, genes associated with ribosome (biogenesis) and protein translation were negative enriched in most clusters. In Tg(Runx1:FLT3^{ITD}IDH2^{R172K}) fish, myeloid and erythroid progenitors showed enrichment of genes associated with cellular proliferation. However, in contrast to Tg(Runx1:FLT3^{ITD}IDH2^{R140Q}) fish, there was depletion of these genes in HSC and HSC-MPP populations. Genes associated with ribosome (biogenesis) and protein translation were enriched in most except T-cell clusters. In both double transgenic fish, genes associated with interferon Y and a were positive enriched in most clusters.

Differential gene expression in specific cell clusters was examined. In Tg(Runx1:FLT3^{ITD}IDH2^{R140Q}) fish, HSC, HSPC-MPP, CMP, myeloid and erythrocyte progenitors showed increased expression of pcna, mcm7 which associated with cell proliferation, cox7c and cox6bl which are associated with metabolism and chac1 and eif4a1a which are associated with Unfolded protein response and down-regulation of ribosomes gene like rps7,rpl14 and rps8a (FIG. 8C-FIG. 8F). In Tg(Runx1:FLT3^{ITD}IDH2^{R172K}) fish, genes associated with IFNγ/α responses, including mhc, uba and isg15 were up-regulated in both immune and myeloid populations, ribosome genes like rps8a, rpl14 and rps7 were also up-regulated (FIG. 8G and FIG. 8H).

Further, subclustering of HSPC-MPP demonstrated expansion of myeloid and lymphoid-associated subclusters in FLT3^{ITD} and IDH2 double mutant zebrafish. Re-clustering the HSPC-MPP population to split between WT, Tg(Runx1:FLT3^{ITD}IDH2^{R140Q}) and Tg(Runx1:FLT3^{ITD}IDH2^{R172k}) cells revealed significant changes in gene expression in Myeloid-1 to -3, Lymphoid-1 to -4, and HSC cells. Tg(Runx1:FLT3^{ITD}IDH2^{R140Q}) cells exhibited changes in 562 genes and Tg(Runx1:FLT3^{ITD}IDH2^{R172K}) exhibited changes in 247 genes compared to 141 genes in WT cells. UMAP feature analysis revealed differences in gradual gene expression of s100a10b, myb, and igic1s1 within HSPC-MPP cell populations. Proportions of the expression distribution between double mutant and WT in the different sub-clusters of HSPC-MPP were quantified. Genes associated with Myeloid-1, Myeloid-2, and Myeloid-3 cells were upregulated and genes associated with Lymphoid-1, Lymphoid-2, Lymphoid-3 and Lymphoid-3 cells were down-regulated for both double mutants compared to the wild-type. The differentially enriched gene ontology (GO) terms within 16 cell clusters between Tg(Runx1:FLT3^{ITD}IDH2^{R140Q}) and WT or between Tg(Runx1:FLT3^{ITD}IDH2^{R172K}) and WT were determined (data not shown).

### Use of Transgenic Fish in Therapeutic Evaluation

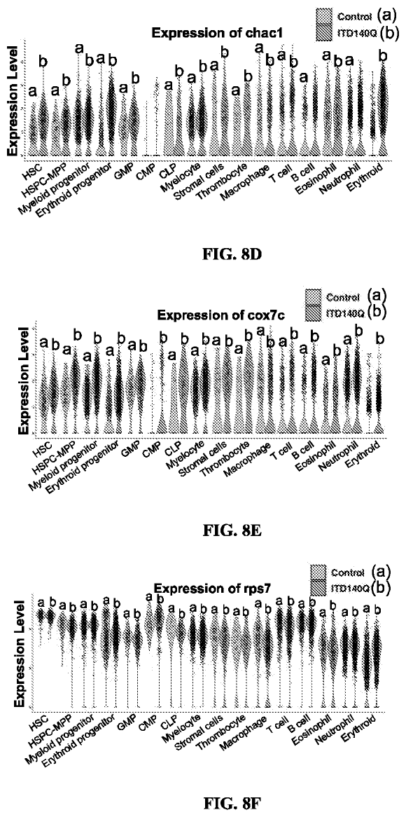

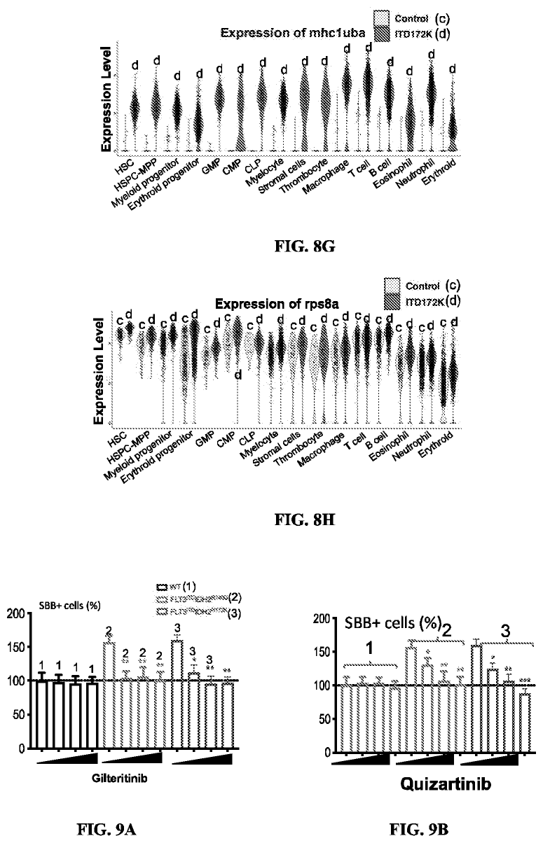

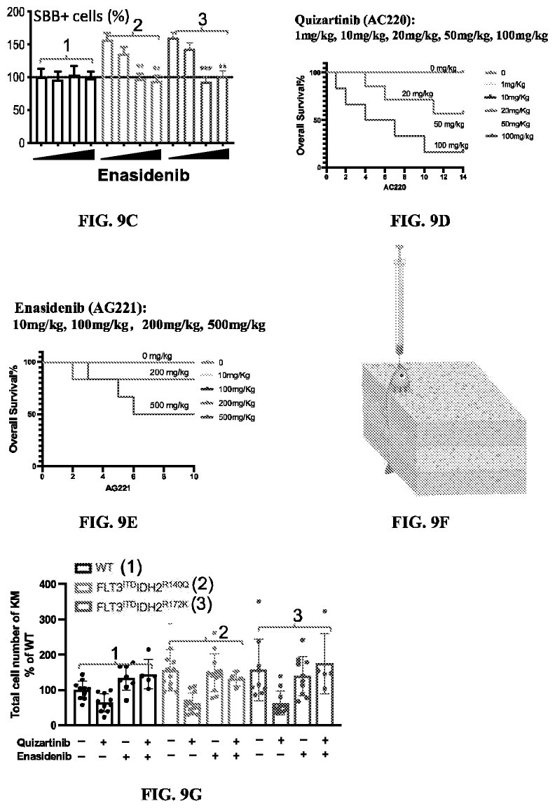

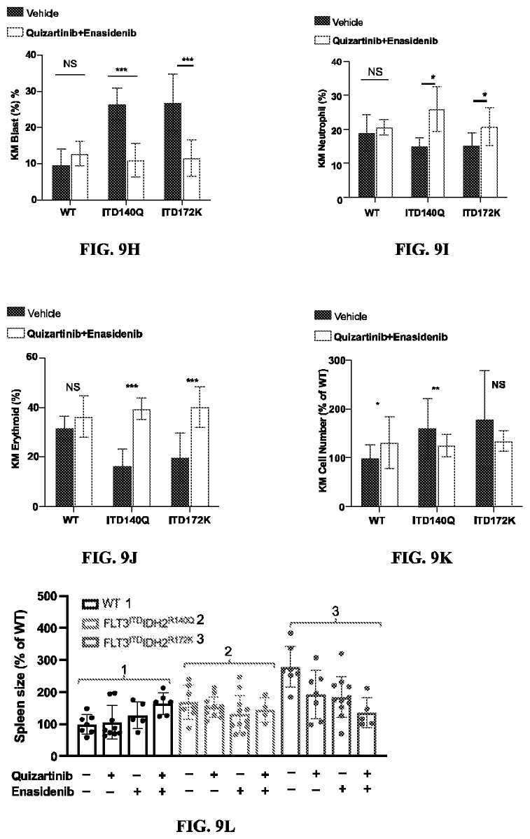

The clinical relevance of the zebrafish models was tested at both embryonic and adult stages. Tg(Runx1:FLT3^{ITD}IDH2^{R172K}) and Tg(Runx1:FLT3^{ITD}IDH2^{R140Q}) embryos and their wildtype siblings were treated with gilteritinib and quizartinib (FLT3 inhibitors) as well as enasidenib (IDH2 inhibitor), which have been shown to induce clinical response and confer survival advantage to patients with FLT3^{ITD} and IDH2 mutations. Using cytochemical staining with SBB as a surrogate for embryonic myelopoiesis, these inhibitors ameliorated the increase in myelopoiesis in the double transgenic embryos (FIG. 9A-FIG. 9C). They were also tested in adult fish. Initial dose-findings studies showed that daily gavage of quizartinib at 10 mg/kg and enasidenib at 100 mg/kg were compatible with normal fish survival (FIG. 9D and FIG. 9E). Double transgenic leukemic fish and their wildtype siblings were treated with 14 days of quizartinib or/and enasidenib (FIG. 9F) and their WKM cellularity, blasts and neutrophil counts were enumerated. Quizartinib but not enasidenib monotherapy significantly reduced cellularity in KM of double transgenic fish (FIG. 9G, middle). Both agents reduced blast counts in KM and induced significant increase in neutrophils and erythroid. Effects of quizartinib and enasidenib combination were tested. The combination significantly reduced blast population (FIG. 9H) restored erythropoiesis (FIG. 9J) and increased neutrophils in KM (FIG. 9I). Cellularity was modestly reduced in Tg(Runx1:FLT3^{ITD}IDH2^{R140Q}) but not in Tg(Runx1:FLT3^{ITD}IDH2^{R171K}) fish (FIG. 9K). Spleen size differed for FLT3^{ITD} and IDH2 double mutant zebrafish following therapeutic treatments. WT zebrafish exhibited increases in spleen size following treatment with Quizartinib and/or Enasidenib (FIG. 9L). While no changes were observed in spleen size of the Tg(Runx1:FLT3^{ITD} IDH2^{R140Q}) double mutant, Tg(Runx1:FLT3^{ITD}IDH2^{R172K}) double mutants had decreased spleen sizes following treatment with Quizartinib and/or Enasidenib (FIG. 9L).

### Discussion

The disclosed studies took advantage of the zebrafish model to examine the unique characteristics of mutation combinations frequently encountered in CN-AML. In this study, zebrafish models of CN-AML were established based on FLT3-ITD and IDH2-R140Q/IDH2-R172K mutation combinations and the data shows that embryonic primitive myelopoiesis was accentuated and full-blown leukemia developed in adult stage. The latter recapitulated the clinical, morphologic, functional, and transcriptomic characteristics of human and mouse AML. Previous studies showed that FLT3-ITD single expression expanded embryonic and adult myelopoiesis but not leukemia (He BLOOD 2014; He EMBO 2020). The observations have not only proven the principle and feasibility of mutation combination models in zebrafish, but it also generated important information that has shed lights to pathogenetic mechanisms of human AML.

First, stable expression of FLT3-ITD and IDH2-R140Q or IDH2-R172K, the two major forms of IDH2 mutations in patients, induced full blown leukemia in adult zebrafish, characterized by circulating blasts, increased KM cellularity and blasts, which were MPO positive, consistent with their myeloid origin. Erythropoiesis was reduced. The blasts showed irregular nuclear contour and amounted to more than 20% of nucleated cells, reaching the cut-off for AML in patients. Interestingly, morphologic features of myelodysplasia were evident, which have also been reported in IDH2 mutated AML patients (Patel et al., Am J Clin Path 2017). Clinically, the double transgenic fish experienced weight loss, splenomegaly, and reduced survival, reminiscent of patients with myeloid neoplasms. Therefore, our observations demonstrated the pathogenetic role of FLT3-ITD and IDH2 co-mutations in human AML.

Second, single cell transcriptome analysis of KM has empowered us to examine lineage development of zebrafish AML in details. The double transgenic fish showed increase in HSC and both lymphoid and myeloid lineages, consistent with multi-lineage hematopoietic expansion by FLT3-ITD and IDH2 mutations. Expression of transgenes by Runxl promoter gave rise to HSC and hence the multi-lineage expansion in the downstream. However, a close examination of the HSPC-MPP revealed priming of myeloid lineage, suggesting leukemogenesis might begin at HSPC-MPP stage in the zebrafish model. Furthermore, gene expression signatures of FLT3-ITD and IDH2-R140Q co-mutations in zebrafish correlated with those arising from RNA-sequencing studies in the corresponding mouse model (PMCID: PMC5413413), attesting to a conserved pathogenetic mechanisms between zebrafish and mouse leukemia.

Third, leukemia generated by FLT3-ITD and IDH2 co-mutations in zebrafish showed substantial self-renewal potential, as demonstrated by their ability to propagate in serial transplantations, a gold standard for the enumeration of leukemia initiation capabilities. In fact, the leukemia became more aggressive in primary and secondary recipients and shortened host survival, suggesting in vivo selection of leukemic clones that were endowed with leukemia initiating activities. The frequencies of leukemia initiating cells (LIS) in zebrafish models would have to be further enumerated.

Information arising from this study has shed important lights to the hitherto undescribed hematopoietic effects of IDH2 mutations. In particular, IDH2-R172K but not IDH2-R140Q, accentuated T-cell development at both embryonic and adult stage. The effects were cell autonomous and IDH2-R172K mutation could be demonstrated in the thymus.

The zebrafish model was of clinical relevance. As a proof-of-principle, both zebrafish embryos and adults carrying FLT3^{ITD}IDH2^{R172K} and FLT3^{ITD}IDH2^{R140Q} mutations responded to quizartinib/gilteritinib and enasidenib, which were effective agents for FLT3-ITD (Cortes Lancet Oncol 2019; Perl NEJM 2019) and IDH2 mutant AML (Stein Blood 2017; Yen Cancer Dis 2017). Specially, enasidenib, in combination with quizartinib, reduced blast count, increased neutrophil count and restored erythropoiesis, reminiscent of its differentiation effects in IDH2 mutated AM patients (Stein Blood 2017). The advantages of zebrafish model have made it uniquely suitable for high throughput drug screening, which is on-going in our laboratory.

In conclusion, the present study generated zebrafish models of AML carrying FLT3^{ITD}IDH2^{R172K} and FLT3^{ITD}IDH2^{R140Q} mutations, recapitulating the morphologic, clinical, transcriptomic and functional characteristics of the corresponding human diseases. These double transgenic fish will become prototype of zebrafish AML models carrying mutation combinations and powerful tool for rapid drug discovery targeting specific drive mutations.

### Example 2. Generation of Stable Transgenic Zebrafish Lines Expressing Human SRSF2^{P95H} and NRAS^{G12D} Mutations

Transgenic zebrafish lines expressing human SRSF2^{P95H} and NRAS^{G12D} mutations (F0), as driven by Runxl promoter, were generated using Tol2 transgenesis. These lines were out-crossed with wildtype (WT) to generate F1. F1 SRSF2^{P95H} mutant were then crossed with F1 NRAS^{G12D} mutant to generate WT, Tg(Runx1:SRSF2^{P95H}), Tg(Runx1:NRAS^{G12D}) and Tg(Runx1:SRSF2^{P95H} NRAS^{G12D}). Expression of mutations was confirmed in embryos and adult kidney marrow (KM) by quantitative RT-PCR and DNA sequencing.

### Kidney Marrow (KM) Collection for Histological Analysis

Zebrafish KM were harvested from 8 months old transgenic or WT fish, dissociated in 0.9×PBS with 5% fetal bovine serum (FBS) by pipetting, and filtered through a 40 μm nylon cell strainer (Corning, NY, USA). The cells were centrifuged at 500 g for 5 minutes, resuspended, filtered and washed with 0.9×PBS with 5% fetal bovine serum (FBS) at least twice. Cytospin preparations were made with 2×10⁵ to 3×10⁵ cells cytocentrifuge (epredia, USA) at 400 RPM for 5 minutes onto the microscope glass slides. The slides were dried at room temperature for more than 12 hours, stained for Wright's stain or non-specific esterase (NSE), and imaged with Nikon DS-Ri2 digital camera (Nikon, Japan) fitted to a Nikon Eclipse Ni-U microscope (Nikon, Japan).

### Blood Collection and Blood Smear

Glass capillary (GC100TF-15, Warner Instruments, USA) was soaked in 5 mg/ml heparin and air-dried at room temperature for at least 1 hour. Adult fish were anesthetized with 0.015% Tricaine in E3 medium and placed onto a dissection board covered with wet paper towels. The blood collection apparatus comprised a heparinized needle, a silicone tubing (0.8 mm ID, Biorad) and a DistriTip microsyringe (125 μl, Gilson). Blood was collected by inserting the glass needle at a 45°-90° angle into the dorsal aorta (Zang et al., 2015) of the zebrafish and 5-10 μl of blood could be drawn at each time to prepare for a blood smear. The slides of the blood smear were stained for Wright's. Cellular morphology was imaged with Nikon DS-Ri2 digital camera fitted to a Nikon Eclipse Ni-U microscope (Nikon, Japan).

### Result

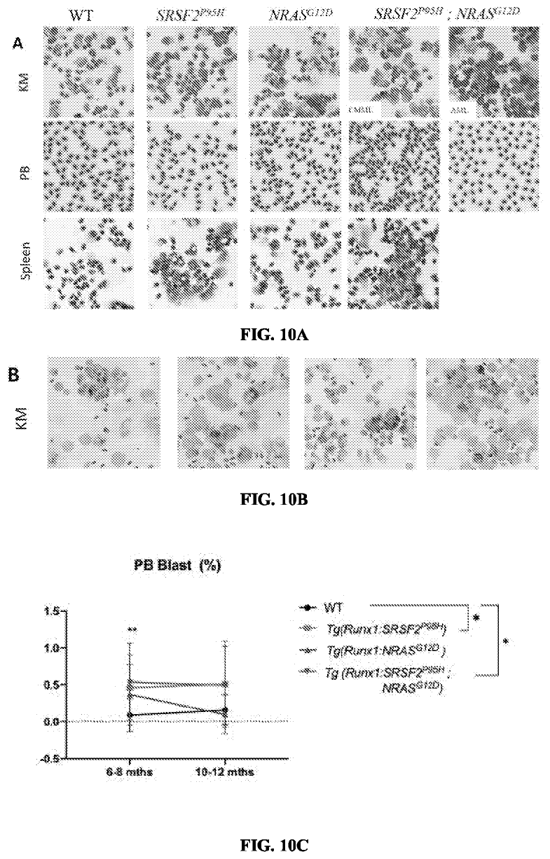

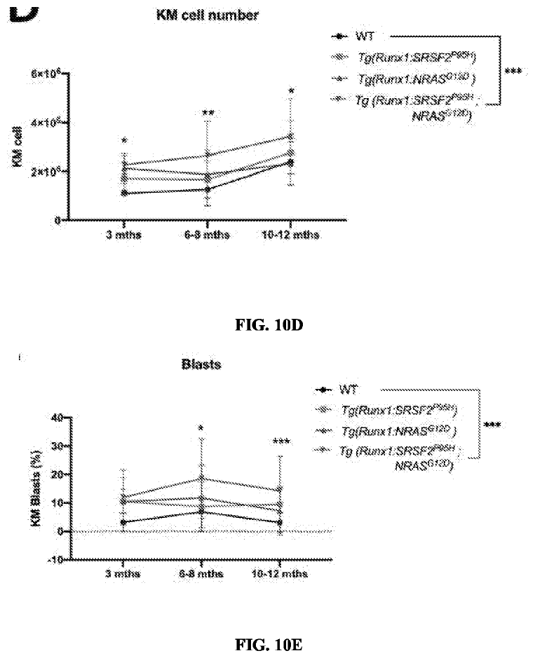

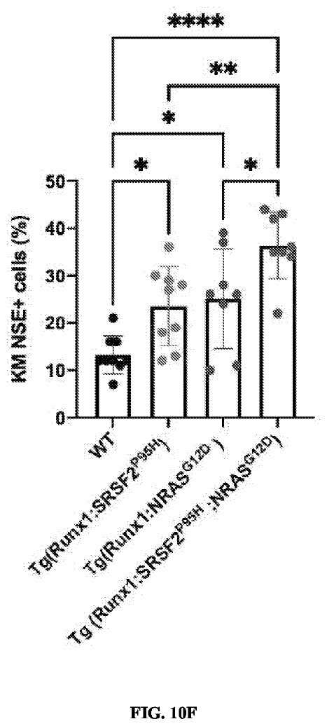

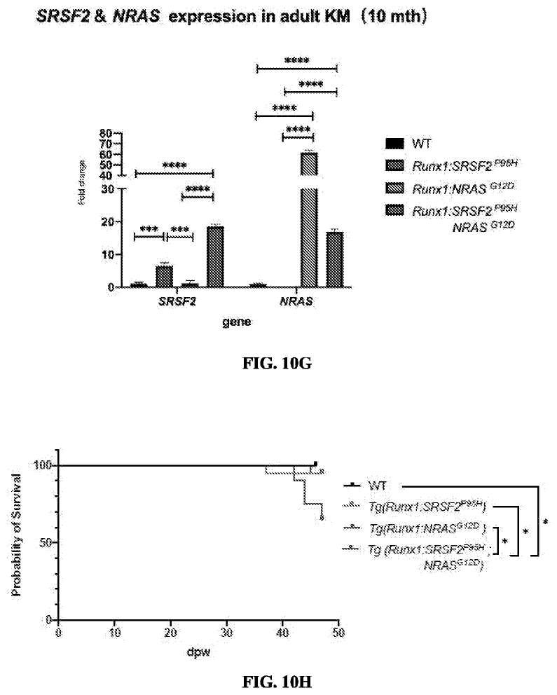

FIG. 10A-10H show development of CMML or AML-like disease in SRSF2 and NRAS mutant zebrafish. FIG. 10A. Representative Wright and Giemsa staining of the peripheral blood (PB), Kidney marrow (KM) cells and spleen cells, and Nonspecific esterase (NSE) staining of the KM cells FIG. 10B) from the mutant zebrafish and WT siblings. FIG. 10C. The percentage of the blast cell in the PB of the transgenic mutant zebrafish and WT siblings. FIG. 10D. The total cell number, the percentage of blast cell (FIG. 10E), the percentage of NSE+ cells (FIG. 10F) in the KM of the transgenic mutant zebrafish compared with WT siblings. FIG. 10F Relative mRNA expression of SRSF2 and NRAS in the KM of the transgenic mutant zebrafish and wildtype zebrafish. H Overall survival of the WT, single mutant, and double mutant. Data are mean±s.e.m. One-way Anova was performed for FIGS. 10C, 10D, 10E, 10F and 10G, *P<0.05, **P<0.01, ****P<0.0001. Log-Rank test was performed for FIG. 10H, *P<0.05. In general, this study showed that zebrafish model of CMML based on co-expression of SRSF2 and NRAS mutations recapitulated the diverse clinical repertoire of this disease, ranging from MDS to MPN and leukemic transformation into AML occurred in some animals.

### Example 3. Generation of Stable Transgenic Zebrafish Lines Expressing Human IDH2 Mutations with Loss of asxl1

Transgenic zebrafish carrying IDH2^{R172K} mutation were generated according to Wang D et al (Wang et al., 2023). These lines were crossed to Tg(asxl1+/−) background (Fang et al., 2021, Leukemia, 35, 2299-2310) to generate Tg(asxl1^{+/-} IDH2^{R172K}). Fish were assigned to different groups according to their genotype. The expression of human IDH2^{R172K} in KM of the mutant fish was confirmed via qPCR.

### Kidney Marrow (KM) Collection for Histologic Analysis

Zebrafish KM were harvested from 8 months old transgenic or WT fish, dissociated in 0.9×PBS with 5% fetal bovine serum (FBS) by pipetting, and filtered through a 40 μm nylon cell strainer (Corning, NY, USA). The cells were centrifuged at 500 g for 5 minutes, resuspended, filtered and washed with 0.9×PBS with 5% fetal bovine serum (FBS) at least twice. Cytospin preparations were made with 2×10⁵ to 3×10⁵ cells cytocentrifuge (epredia, USA) at 400 RPM for 5 minutes onto the microscope glass slides. The slides were dried at room temperature for more than 12 hours, stained for Wright's stain or non specific esterase (NSE), and imaged with Nikon DS-Ri2 digital camera (Nikon, Japan) fitted to a Nikon Eclipse Ni-U microscope (Nikon, Japan).

### Blood Collection and Blood Smear

Glass capillary (GC100TF-15, Warner Instruments, USA) was soaked in 5 mg/ml heparin and air-dried at room temperature for at least 1 hour. Adult fish were anesthetized with 0.015% Tricaine in E3 medium and placed onto a dissection board covered with wet paper towels. The blood collection apparatus comprised a heparinized needle, a silicone tubing (0.8 mm ID, Biorad) and a DistriTip microsyringe (125 μl, Gilson). Blood was collected by inserting the glass needle at a 45990° angle into the dorsal aorta (Zang et al., 2015, J Vis Exp, e53272.) of the zebrafish and 5-10 μl of blood could be drawn at each time to prepare for a blood smear. The slides of the blood smear were stained for Wright's. Cellular morphology was imaged with Nikon DS-Ri2 digital camera fitted to a Nikon Eclipse Ni-U microscope (Nikon, Japan).

### Result

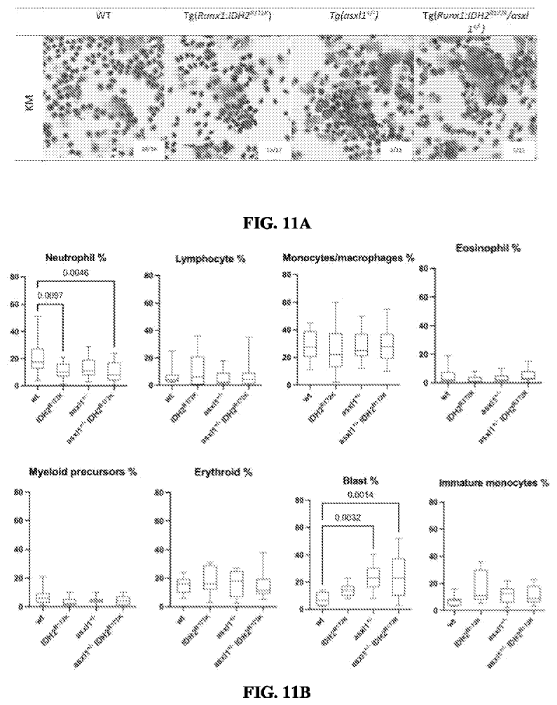

FIGS. 11A and 11B show cytospin analysis of 9-month-old transgenic line carrying asxl1 and IDH2^{R172K} mutations. (FIG. 11A) Representative Wright-Giemsa staining of cytospin of kidney marrow (KM) of the control, single and double mutant transgenic line. (FIG. 11B) The percentage of the erythroid, neutrophils, lymphocytes monocytes/macrophages, eosinophil, myeloid precursors, immature monocytes and blast-like cells from the KM was quantified. Statistical analysis was performed using the Student's t test, p<0.05 was considered to be statistically significant. In conclusion, the present study generated zebrafish models of AML carrying asxl1+/− and IDH2^{R172K} mutations, recapitulating the morphologic characteristics of the corresponding human diseases. These double transgenic fish will become prototypes of zebrafish AML models carrying mutation combinations and powerful tools for rapid drug discovery targeting specific drive mutations.

### Example 4. Generation of Stable Transgenic Zebrafish Lines Expressing Human SRSF2^{P95H} and Loss of Asxl1

Transgenic zebrafish expressing human SRSF2^{P95H} [Tg(Runx1:SRSF2^{P95H})] in hematopoietic stem and progenitor cells, driven by Runxl enhancer and mouse β-globin minimal promoter, were generated using Tol2 transgenesis. Successful transgenesis was shown by EGFP (driven by cmlc2) expression in the developing heart and confirmed by PCR genotyping. Adult zebrafish were outcrossed with WT (TU) to confirm germline transmission to F1. These lines were crossed to Tg(asxl1^{+/-}) background (Fang et al., 2021, Leukemia, 35, 2299-2310) to generate Tg(asxl1^{+/-} SRSF2^{P95H}). Fish were assigned to different groups according to their genotype. The expression of human SRSF2 in KM of the mutant fish was confirmed via qPCR.

### Kidney Marrow (KM) Collection for Histologic Analysis

Zebrafish KM were harvested from 8 months old transgenic or WT fish, dissociated in 0.9×PBS with 5% fetal bovine serum (FBS) by pipetting, and filtered through a 40 μm nylon cell strainer (Corning, NY, USA). The cells were centrifuged at 500 g for 5 minutes, resuspended, filtered and washed with 0.9×PBS with 5% fetal bovine serum (FBS) at least twice. Cytospin preparations were made with 2×10⁵ to 3×10⁵ cells cytocentrifuge (epredia, USA) at 400 RPM for 5 minutes onto the microscope glass slides. The slides were dried at room temperature for more than 12 hours, stained for Wright's stain or nonspecific esterase (NSE), and imaged with Nikon DS-Ri2 digital camera (Nikon, Japan) fitted to a Nikon Eclipse Ni-U microscope (Nikon, Japan).

### Blood Collection and Blood Smear

Glass capillary (GC100TF-15, Warner Instruments, USA) was soaked in 5 mg/ml heparin and air-dried at room temperature for at least 1 hour. Adult fish were anesthetized with 0.015% Tricaine in E3 medium and placed onto a dissection board covered with wet paper towels. The blood collection apparatus comprised a heparinized needle, a silicone tubing (0.8 mm ID, Biorad) and a DistriTip microsyringe (125 μl, Gilson). Blood was collected by inserting the glass needle at a 45290° angle into the dorsal aorta (Zang et al., 2015, J Vis Exp, e53272) of the zebrafish and 5-10 μl of blood could be drawn at each time to prepare for a blood smear. The slides of the blood smear were stained for Wright's. Cellular morphology was imaged with Nikon DS-Ri2 digital camera fitted to a Nikon Eclipse Ni-U microscope (Nikon, Japan).

### Result

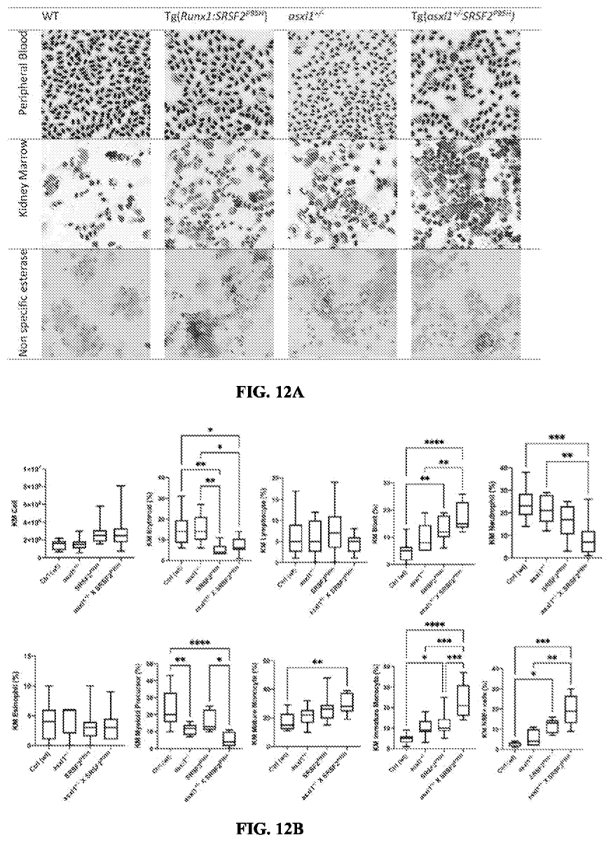

FIGS. 12A and 12B show cytospin analysis of 6-month-old transgenic line carrying asxl1+/− and SRSF2P95H mutations. (FIG. 12A) Representative Wright-Giemsa staining of peripheral blood smear, cytospin of kidney marrow, and nonspecific esterase staining (NSE) of kidney marrow of the control, single and double mutant transgenic line. (FIG. 12B) The percentage of the erythroid, neutrophils, lymphocytes, blast-like cells, myeloid precursors, eosinophils, mature monocytes, immature monocytes, and NSE+ cells from the KM was quantified. Statistical analysis was performed using the Student's t test, p<0.05 was considered to be statistically significant. In conclusion, our findings suggested that asxl1 and SRSF2 mutation combination induced leukemogenesis in zebrafish with variable penetrance representing both the chronic phase and blastic transformation, reminiscent of disease progression of human CMML. The model can be used to shed light to the pathogenetic mechanisms of aberrant epigenetic modifications and splicing in CMML.

### Example 5. Generation of Stable Transgenic Zebrafish Lines Expressing Human Mutations of SRSF2^{P95H} and FLT3^{ITD}

Transgenic zebrafish expressing human SRSF2^{P95H} [Tg(Runx1:SRSF2^{P95H})] in hematopoietic stem and progenitor cells, driven by Runx1 enhancer and mouse β-globin minimal promoter, were generated using Tol2 transgenesis. Successful transgenesis was shown by EGFP (driven by cmlc2) expression in the developing heart and confirmed by PCR genotyping. Adult zebrafish were outcrossed with WT (TU) to confirm germline transmission to F1. These lines were crossed to Tg(Runx1:FLT3^{ITD}) background (He et al., 2020, EMBO Mol Med, 12, e10895.) to generate Tg(Runx1:FLT3^{ITD}SRSF2^{P95H}). Fish were assigned to different groups according to their genotype. The expression of human FLT3 and SRSF2 in KM of the mutant fish was confirmed via qPCR.

### Kidney Marrow (KM) Collection for Histologic Analysis

Zebrafish KM were harvested from 8 months old transgenic or WT fish, dissociated in 0.9×PBS with 5% fetal bovine serum (FBS) by pipetting, and filtered through a 40 μm nylon cell strainer (Corning, NY, USA). The cells were centrifuged at 500 g for 5 minutes, resuspended, filtered and washed with 0.9×PBS with 5% fetal bovine serum (FBS) at least twice. Cytospin preparations were made with 2×10⁵ to 3×10⁵ cells cytocentrifuge (epredia, USA) at 400 RPM for 5 minutes onto the microscope glass slides. The slides were dried at room temperature for more than 12 hours, stained for Wright's stain or non-specific esterase (NSE), and imaged with Nikon DS-Ri2 digital camera (Nikon, Japan) fitted to a Nikon Eclipse Ni-U microscope (Nikon, Japan).

### Blood Collection and Blood Smear

Glass capillary (GC100TF-15, Warner Instruments, USA) was soaked in 5 mg/ml heparin and air-dried at room temperature for at least 1 hour. Adult fish were anesthetized with 0.015% Tricaine in E3 medium and placed onto a dissection board covered with wet paper towels. The blood collection apparatus comprised a heparinized needle, a silicone tubing (0.8 mm ID, Biorad) and a DistriTip microsyringe (125 μl, Gilson). Blood was collected by inserting the glass needle at a 45°-90° angle into the dorsal aorta (Zang et al., 2015) of the zebrafish and 5-10 μl of blood could be drawn at each time to prepare for a blood smear. The slides of the blood smear were stained for Wright's. Cellular morphology was imaged with Nikon DS-Ri2 digital camera fitted to a Nikon Eclipse Ni-U microscope (Nikon, Japan).

### Result

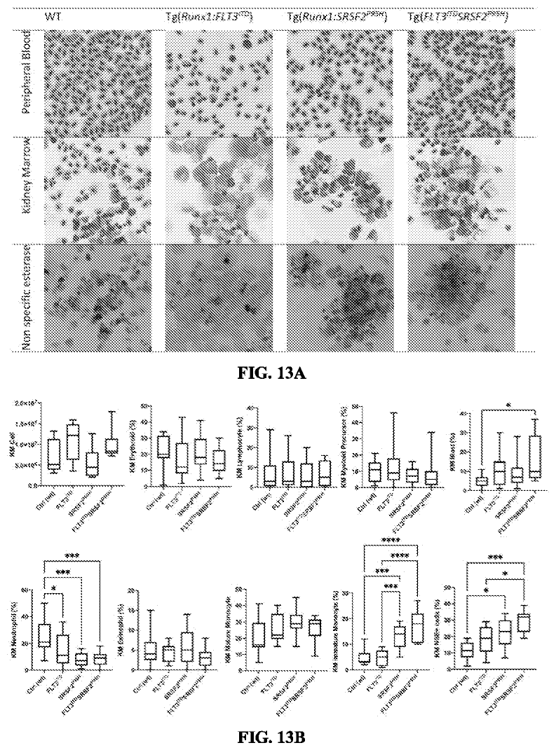

FIGS. 13A and 13B show cytospin analysis of 1-year-old transgenic line carrying FLT3ITD and SRSF2P95H mutations. (FIG. 13A) Representative Wright-Giemsa staining of peripheral blood smear, cytospin of kidney marrow, and nonspecific esterase staining (NSE) of kidney marrow of the control, single and double mutant transgenic line. (FIG. 13B) The percentage of the erythroid, neutrophils, lymphocytes, blast-like cells, myeloid precursors, eosinophils, mature monocytes, immature monocytes, and NSE+ cells from the KM was quantified. Statistical analysis was performed using the Student's t test, p<0.05 was considered to be statistically significant. Taken together, our findings demonstrated that FLT3^{ITD} cooperates with SRSF2^{P95H} to drive the initiation and progression of CMML and the subsequent transformation to AML.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

1. A genetically modified zebrafish model of human acute myeloid leukemia (AML), wherein the zebrafish stably expresses genes comprising least two human gene mutations selected from the group consisting of FLT3^{ITD}, NPMc+, DNMT3A^{R882}, NRAS^{G12D}, IDH2^{R140Q}, IDH2^{R172K}, TET2^{-/-}, RUNX1^{-/-}, IDH1^{R132H}, WT1^{-/-}, TP53^{-/-}, SRSF2^{P95H}, ASXL^{-/-}, STAG2^{-/-}, and PHF6^{-/-}.

2. The genetically modified zebrafish of claim 1, wherein the zebrafish carries the specific mutation combinations FLT3^{ITD}, IDH2^{R140Q} and/or IDH2^{R172K}.

3. The genetically modified zebrafish of claim 1, (a) wherein the zebrafish carries the specific mutation combinations: (a) FLT3^{ITD} and IDH2^{R140Q}, (b) FLT3^{ITD} and IDH2^{R172K}; (c) SRSF2^{P95H} NRAS^{G12D}; (d). asxl1^{+/-} IDH2^{R172K}; (e) FLT3^{ITD}SRSF2^{P95H}; or (f) asxl1^{+/-} SRSF2^{P95H} and/or further comprising a zebrafish specific expression sequence operably linked to a nucleic acid sequence encoding a fluorescent reporter polypeptide.

4. (canceled)

5. The genetically modified zebrafish of claim 4, wherein the fluorescent reporter polypeptide is selected from the group consisting of green fluorescent protein (GFP), green reef coral fluorescent protein (G-RCFP), cyan fluorescent protein (CFP), red fluorescent protein (RFP or dsRed2), blue fluorescent protein (BFP) and yellow fluorescent protein (YFP).

6. The genetically modified zebrafish of claim 3, comprising a zebrafish expression sequence, wherein the zebrafish specific expression sequence is a neutrophil-specific promoter, macrophage-specific promoter, erythroid cell specific promoter, effector cell specific promoter or leukocyte promoter.

7. The genetically modified zebrafish of claim 1 comprising: mpo:EGFP, gata1: RFP, Runx1:FLT3^{ITD}IDH2^{R172K/R140Q} or lyz:EGFP, corola:DsRED, Runx1:FLT3^{ITD}IDH2^{R172K/R140Q}; or Runx1:FLT3^{ITD}SRSF2^{P95H}; Runx1:SRSF2^{P95H} NRAS^{G12D}.

8. The genetically modified zebrafish of claim 1, wherein the genetically modified zebrafish produces an expression product, wherein expression of the expression product is transmitted through the germline.

9. The genetically modified zebrafish of claim 6, wherein the human zebrafish expression sequence and the nucleic acid sequence encoding the fluorescent reporter are contained in an exogenous construct.

10. The genetically modified zebrafish of claim 9, wherein the construct further comprises (a) intron sequences operably linked to the nucleic acid sequence encoding the fluorescent reporter, (b) a polyadenylation signal operably linked to the nucleic acid sequence encoding the fluorescent reporter, or both.

11. The genetically modified zebrafish of claim 1, wherein the genetically modified zebrafish is developed from, or is the progeny of a genetically modified zebrafish developed from, an embryonic cell into which the exogenous construct was introduced.

12. The genetically modified zebrafish of claim 1, wherein the zebrafish carries the specific mutation combinations: (a) FLT3^{ITD} and NPMc+, (b) FLT3^{ITD} and DNMT3A^{R882}, or (c) FLT3^{ITD}, NPMc+, and DNMT3A^{R882}.

13. The genetically modified zebrafish of claim 1, wherein the zebrafish carries the specific mutation combinations: (a) FLT3^{ITD} and NRAS^{G12D} (b) FLT3^{ITD} and IDH2^{R140Q}, or (c) FLT3^{ITD}, NRAS^{G12D} and IDH2^{R140Q}.

14. The genetically modified zebrafish of claim 1, wherein the zebrafish carries the specific mutation combinations: FLT3^{ITD}, NPMc+, DNMT3A^{R882}, NRAS^{G12D}, IDH2^{R140Q}, IDH2^{R172K}, TET2^{-/-}, RUNX1^{-/-}, IDH1^{R132H}, WT1^{-/-}, TP53^{-/-}, SRSF2^{P95H}, ASXL^{-/-}, STAG2^{-/-}, and PHF6^{-/-}.

15. A method of using the genetically modified zebrafish of claim 1 for drug screening comprising contacting the genetically modified zebrafish with a test agent, at test concentrations and test intervals to determine the therapeutic effect if any, of the test agent.

16. The method of claim 15, wherein the zebrafish are embryos, larvae, juveniles or adults.

17. A method of identifying a candidate therapeutic compound for the treatment of AML, the method comprising: contacting the genetically modified zebrafish of claim 1 with a test compound and determining the effect of the agent on a phenotype selected from the group consisting of survival, spleen size, decrease in myelopoiesis, erythropoiesis, whole kidney marrow cellularity, blasts and neutrophil counts.

18. The method of claim 17, wherein the genetically modified zebrafish is an embryo.

19. The method of claim 17, wherein the genetically modified zebrafish is an adult fish.

20. The method of claim 17, wherein the genetically modified zebrafish is at the larval stage.

21. The method of any one of claim 17, wherein detection of an improvement in the phenotype indicates identification of an agent that can be used in the treatment of AML.
